SEQUENCE LISTING (89 sequences)

SEQ ID NO: 1 - protein, 276 aa, synthetic construct [sequence omitted]

SEQ ID NO: 2 - protein, 275 aa, synthetic construct [sequence omitted]

SEQ ID NO: 3 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 4 - protein, 99 aa, Homo sapiens
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 5 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, synthetic construct
DKTHTCPPCPAPEAALLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 5 aa, synthetic construct
GCGGS

SEQ ID NO: 10 - protein, 10 aa, synthetic construct
GCGGSGGGGS

SEQ ID NO: 11 - protein, 15 aa, synthetic construct
GCGGSGGGGSGGGGS

SEQ ID NO: 12 - protein, 20 aa, synthetic construct
GCGGSGGGGSGGGGSGGGGS

SEQ ID NO: 13 - protein, 25 aa, synthetic construct
GCGGSGGGGSGGGGSGGGGSGGGGS

SEQ ID NO: 14 - protein, 30 aa, synthetic construct
GCGGSGGGGSGGGGSGGGGSGGGGSGGGGS

SEQ ID NO: 15 - protein, 35 aa, synthetic construct
GCGGSGGGGSGGGGSGGGGSGGGGSGGGGSGGGGS

SEQ ID NO: 16 - protein, 40 aa, synthetic construct
GCGGSGGGGSGGGGSGGGGSGGGGSGGGGSGGGGSGGGGS

SEQ ID NO: 17 - protein, 45 aa, synthetic construct
GCGGSGGGGSGGGGSGGGGSGGGGSGGGGSGGGGSGGGGSGGGGS

SEQ ID NO: 18 - protein, 50 aa, synthetic construct
GCGGSGGGGSGGGGSGGGGSGGGGSGGGGSGGGGSGGGGSGGGGSGGGGS

SEQ ID NO: 19 - protein, 55 aa, synthetic construct
GCGGSGGGGSGGGGSGGGGSGGGGSGGGGSGGGGSGGGGSGGGGSGGGGSGGGGS

SEQ ID NO: 20 - protein, 5 aa, synthetic construct
CGGGS

SEQ ID NO: 21 - protein, 55 aa, synthetic construct
  VARIANT 6..55: note = Wherein up to 9 copies of GGGGS can be deleted
CGGGSGGGGSGGGGSGGGGSGGGGSGGGGSGGGGSGGGGSGGGGSGGGGSGGGGS

SEQ ID NO: 22 - protein, 10 aa, synthetic construct
CGGGSGGGGS

SEQ ID NO: 23 - protein, 15 aa, synthetic construct
CGGGSGGGGSGGGGS

SEQ ID NO: 24 - protein, 20 aa, synthetic construct
CGGGSGGGGSGGGGSGGGGS

SEQ ID NO: 25 - protein, 25 aa, synthetic construct
CGGGSGGGGSGGGGSGGGGSGGGGS

SEQ ID NO: 26 - protein, 30 aa, synthetic construct
CGGGSGGGGSGGGGSGGGGSGGGGSGGGGS

SEQ ID NO: 27 - protein, 35 aa, synthetic construct
CGGGSGGGGSGGGGSGGGGSGGGGSGGGGSGGGGS

SEQ ID NO: 28 - protein, 40 aa, synthetic construct
CGGGSGGGGSGGGGSGGGGSGGGGSGGGGSGGGGSGGGGS

SEQ ID NO: 29 - protein, 45 aa, synthetic construct
CGGGSGGGGSGGGGSGGGGSGGGGSGGGGSGGGGSGGGGSGGGGS

SEQ ID NO: 30 - protein, 50 aa, synthetic construct
CGGGSGGGGSGGGGSGGGGSGGGGSGGGGSGGGGSGGGGSGGGGSGGGGS

SEQ ID NO: 31 - protein, 55 aa, synthetic construct; identical to SEQ ID NO: 21

SEQ ID NO: 32 - protein, 5 aa, synthetic construct
GSGGS

SEQ ID NO: 33 - protein, 4 aa, synthetic construct
GGGS

SEQ ID NO: 34 - protein, 4 aa, synthetic construct
GGSG

SEQ ID NO: 35 - protein, 5 aa, synthetic construct
GGSGG

SEQ ID NO: 36 - protein, 5 aa, synthetic construct
GSGSG

SEQ ID NO: 37 - protein, 5 aa, synthetic construct
GSGGG

SEQ ID NO: 38 - protein, 5 aa, synthetic construct
GGGSG

SEQ ID NO: 39 - protein, 5 aa, synthetic construct
GSSSG

SEQ ID NO: 40 - protein, 41 aa, synthetic construct
  VARIANT 2..41: note = Where up to 9 copies of SSSS can be deleted
GSSSSSSSSSSSSSSSSSSSSSSSSSSSSSSSSSSSSSSSS

SEQ ID NO: 41 - protein, 5 aa, synthetic construct
GGGGS

SEQ ID NO: 42 - protein, 10 aa, synthetic construct
GGGGSGGGGS

SEQ ID NO: 43 - protein, 15 aa, synthetic construct
GGGGSGGGGSGGGGS

SEQ ID NO: 44 - protein, 20 aa, synthetic construct
GGGGSGGGGSGGGGSGGGGS

SEQ ID NO: 45 - protein, 25 aa, synthetic construct
GGGGSGGGGSGGGGSGGGGSGGGGS

SEQ ID NO: 46 - protein, 30 aa, synthetic construct
GGGGSGGGGSGGGGSGGGGSGGGGSGGGGS

SEQ ID NO: 47 - protein, 35 aa, synthetic construct
GGGGSGGGGSGGGGSGGGGSGGGGSGGGGSGGGGS

SEQ ID NO: 48 - protein, 40 aa, synthetic construct
GGGGSGGGGSGGGGSGGGGSGGGGSGGGGSGGGGSGGGGS

SEQ ID NO: 49 - protein, 45 aa, synthetic construct
GGGGSGGGGSGGGGSGGGGSGGGGSGGGGSGGGGSGGGGSGGGGS

SEQ ID NO: 50 - protein, 50 aa, synthetic construct
  VARIANT 1..50: note = Wherein up to 9 copies of GGGGS can be deleted
GGGGSGGGGSGGGGSGGGGSGGGGSGGGGSGGGGSGGGGSGGGGSGGGGS

SEQ ID NO: 51 - protein, 5 aa, synthetic construct
AAAGG

SEQ ID NO: 52 - protein, 20 aa, synthetic construct
GSSSSGSSSSGSSSSGSSSS

SEQ ID NO: 53 - protein, 25 aa, synthetic construct
GSSSSGSSSSGSSSSGSSSSGSSSS

SEQ ID NO: 54 - protein, 133 aa, Homo sapiens [sequence omitted]

SEQ ID NO: 55 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 56 - protein, 55 aa, synthetic construct; identical to SEQ ID NO: 19
  VARIANT 6..55: note = Wherein up to 9 copies of GGGGS can be deleted

SEQ ID NO: 57 - protein, 50 aa, synthetic construct; identical to SEQ ID NO: 18
  VARIANT 6..50: note = Wherein up to 8 copies of GGGGS can be deleted

SEQ ID NO: 58 - protein, 812 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 123 aa, synthetic construct
VLDFAPPGAGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 60 - protein, 9 aa, synthetic construct
VLDFAPPGA

SEQ ID NO: 61 - protein, 276 aa, synthetic construct
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 62 - protein, 276 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 63 - protein, 276 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 64 - protein, 276 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 65 - protein, 276 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 66 - protein, 276 aa, synthetic construct
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 67 - protein, 276 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 68 - protein, 276 aa, synthetic construct
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 69 - protein, 276 aa, synthetic construct [sequence omitted]

SEQ ID NO: 70 - protein, 276 aa, synthetic construct
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 71 - protein, 276 aa, synthetic construct
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 72 - protein, 276 aa, synthetic construct
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 73 - protein, 276 aa, synthetic construct
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 74 - protein, 276 aa, synthetic construct
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 75 - protein, 276 aa, synthetic construct
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 76 - protein, 276 aa, synthetic construct
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 77 - protein, 276 aa, synthetic construct
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 78 - protein, 276 aa, synthetic construct
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 79 - protein, 276 aa, synthetic construct
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 80 - protein, 276 aa, synthetic construct
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 81 - protein, 276 aa, synthetic construct
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 82 - protein, 276 aa, synthetic construct
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 83 - protein, 276 aa, synthetic construct
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 84 - protein, 276 aa, synthetic construct
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 85 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 86 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 87 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 88 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 89 - protein, 50 aa, synthetic construct; identical to SEQ ID NO: 50

(19) United States
(12) Patent Application Publication
(10) Pub. No.: US 2024/0368246 A1
(43) Pub. Date: Nov. 7, 2024

(54) MULTIMERIC T-CELL MODULATORY POLYPEPTIDES AND METHODS OF USE THEREOF

(71) Applicant: Cue Biopharma, Inc., Bostson, MA (US)

(72) Inventors: Kenneth James Pienta, Boston, MA (US); Matteo G. Levisetti, Boston, MA (US); Steven Paul Margossian, Boston, MA (US)

(21) Appl. No.: 18/653,712

(22) Filed: May 2, 2024

Related U.S. Application Data
(63) Continuation of application No. PCT/US2022/079169, filed on Nov. 2, 2022.
(60) Provisional application No. 63/275,662, filed on Nov. 4, 2021.
(60) Provisional application No. 63/400,982, filed on Aug. 25, 2022.

(51) Int. Cl. C07K 14/74; A61K 38/00; A61P 35/00; A61P 37/04; C07K 14/55

(52) U.S. Cl. CPC C07K 14/70539; A61P 35/00; A61P 37/04; C07K 14/55; A61K 38/00; C07K 2319/30; C07K 2319/81

(57) ABSTRACT

The present disclosure provides T-cell modulatory multimeric polypeptides (T.M.MPs) that comprise an immunomodulatory polypeptide and that comprise an epitope-presenting Wilms tumor-1 (WT-1) peptide. A TMMP is useful for modulating the activity of a T cell. and for modulating an immune response in an individual, e.g., for the treatment of a cancer associated with WT-1.

### CROSS-REFERENCE

This application claims the benefit of U.S. Provisional Patent Application No. 63/275,662, filed Nov. 4, 2021 and U.S. Provisional Patent Application No. 63/400,982, filed Aug. 25, 2022, which applications are incorporated herein by reference in their entirety.

### INCORPORATION-BY-REFERENCE OF MATERIAL ELECTRONICALLY SUBMITTED

A Sequence Listing is provided herewith as a Sequence Listing XML, “CUEB-148WO_SEQ_LIST” created on Oct. 13, 2022 and having a size of 90 KB. The contents of the Sequence Listing XML are incorporated by reference herein in their entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals-epitope/TCR binding and engagement of APC costimulatory proteins with T cell costimulatory proteins-are required to drive T cell specificity and activation or inhibition. The TCR is specific for a given epitope; however, the costimulatory protein not epitope specific and instead is generally expressed on all T cells or on large T cell subsets.

### SUMMARY

The present disclosure provides T-cell modulatory multimeric polypeptides (TMMPs) that comprise an immunomodulatory polypeptide and an epitope-presenting Wilms tumor-1 (WT-1) peptide. A TMMP (also known as a “synTac” or an “Immuno-STAT”) is useful for modulating the activity of a T cell, and for modulating an immune response in an individual, e.g., for treating a cancer associated with WT-1.

### BRIEF DESCRIPTION OF THE DRAWINGS

FIG. 1 is a schematic depiction of the polypeptides 1715Δ and 2380 of the present disclosure.

FIG. 2A and 2B provide the amino acid sequences of polypeptides 1715Δ (SEQ ID NO: 58) and 2380 (SEQ ID NO:59). Additional sequences: AAAGG (SEQ ID NO:51), VLDFAPPGA (SEQ ID NO:60). The polypeptide 1715Δ is 1715 without the C-terminal Lys.

FIG. 3A and 3B provide a schematic depiction of a double disulfide-linked TMMP comprising polypeptides 1715Δ and 2380 (FIG. 3A), and a TMMP that is a dimer comprising two TMMPs comprising polypeptides 1715Δ and 2380 (FIG. 3B).

FIG. 4 provides an alignment of HLA-A heavy chain amino acid sequences (SEQ ID NOs: 61, 1, 62-68, respectively).

FIG. 5 provides an alignment of HLA-B heavy chain amino acid sequences (SEQ ID NOs: 69-75, respectively).

FIG. 6 provides an alignment of HLA-C heavy chain amino acid sequences (SEQ ID NOs: 76-84, respectively).

FIG. 7A-7D provide the amino acid sequences of HLA-E heavy chains, where FIG. 7A provides the amino acid sequence of HLA-E*01:01 (wild-type; SEQ ID NO:85); FIG. 7B provides the amino acid sequence of HLA-E*01:01 with Y84C and A2346C substitutions (SEQ ID NO:86); FIG. 7C provides the amino acid sequence of HLA-E*01:03 (wild-type; SEQ ID NO:87); and FIG. 7D provides the amino acid sequence of HLA-E*01:03 with Y84C and A2346C substitutions (SEQ ID NO:88).

FIG. 8 depicts expansion of WT1₃₇₋₄₅-specific CD8⁺ T cells from unprimed PBMCs, in which the expansion was induced by a TMMP of the present disclosure (“CUE-102/A02 WT1₃₇₋₄₅ IST”).

FIG. 9A-9B depict expansion of WT1₃₇₋₄₅-specific CD8⁺ T cells from WT1₃₇₋₄₅ peptide-primed PBMCs, in which the expansion was induced by a TMMP of the present disclosure (“CUE-102/A02 WT1₃₇₋₄₅ IST”).

FIG. 10A-10B depict production of TNF-α, IL-6, and IFN-γ, and upregulation of CD69, induced by a TMMP of the present disclosure (“CUE-102/A02 WT1₃₇₋₄₅IST”) or by wild-type IL-2.

FIG. 11A-11B depict CTL activity, against peptide-presenting target cells, of WT1₃₇₋₄₅ peptide-specific CD8⁺ T cells expanded from peptide-primed PBMCs in the presence of a TMMP of the present disclosure (“CUE-102/A02 WT1₃₇₋₄₅ IST”).

FIG. 12 depicts the effect of a dimer comprising two TMMPs comprising polypeptides 1715Δ and 2380 (containing the WT1 peptide epitope 37-45), i.e., CUE-102 (or “CUE-102/A02”) on antigen-specific CD8⁺ T cell expansion in naïve HLA-A2 (AAD) transgenic mice.

FIG. 13A-13C depict the effect of a composition comprising CUE-102 on antigen-specific CD8⁺ T cell expansion in naïve HLA-A2 (AAD) transgenic mice.

FIG. 14A-14C depict the effect of a composition comprising CUE-102 on antigen-specific CD8⁺ T cell expansion in naïve HLA-A2 (AAD) transgenic mice given weekly doses for three weeks, followed by a booster dose three weeks later.

FIG. 15A-15B depict the effect of a composition comprising CUE-102 on antigen-specific CD8⁺ T cell expansion in naïve HLA-A2 (AAD) transgenic mice given weekly doses for three weeks, followed by one-week rest, and then weekly doses for an additional three weeks.

FIG. 16 shows that the majority of WT1₃₇₋₄₅-specific CD8⁺ T cells (tetramer+) isolated from the spleens of naïve HLA-A2 transgenic mice in immunized with 4 doses of CUE-102 produced IFNγ, TNFα, CD107a and Granzyme B in response to ex vivo restimulation.

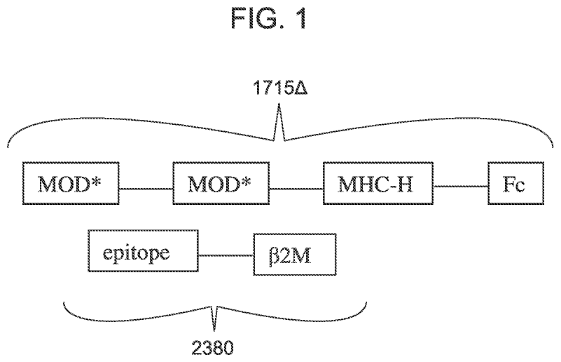

### DEFINITIONS

The terms “polynucleotide” and “nucleic acid.” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide.” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if an MHC class I polypeptide is altered by the addition of one amino acid at the N-terminus, then a reference to position 84 or a specific residue at position 84, will be understood to indicate the amino acids that are at position 85 on the altered polypeptide. Likewise, a reference herein to substitution of a specific amino acid at a specific position, e.g., Y84, is understood to refer to a substitution of an amino acid for the amino acid at position 84 in the wild-type polypeptide. A Y84C substitution is thus understood to be a substitution of Cys residue for the Tyr residue that is present in the wild-type sequence. If, e.g., the wild-type polypeptide is altered to change the amino acid at position 84 from its wild-type amino acid to an alternate amino acid, then the substitution for the amino acid at position 84 will be understood to refer to the substitution for the alternate amino acid. If in such case the polypeptide is also altered by the addition or deletion of one or more amino acids, then the reference to the substitution will be understood to refer to the substitution for the alternate amino acid at the altered position number. A reference to a “non-naturally occurring Cys residue” in a polypeptide, e.g., an MHC class I polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10. Unless otherwise stated, for this disclosure sequence identity is determined using the BLAST computer program.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to as a “MOD”) as used herein refers to an IL-2 variant polypeptide as described herein. A MOD specifically binds a cognate co-immunomodulatory polypeptide (or “co-MOD”) on a T cell, in this case IL-2 receptor or “IL-2R”.

As used herein the term “in vivo” refers to any process or procedure occurring inside of the body.

As used herein, “in vitro” refers to any process or procedure occurring outside of the body.

As used herein, the term “affinity” refers to the equilibrium constant for the reversible binding of two agents (e.g., an antibody and an antigen) and is expressed as a dissociation constant (K_{D}). As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding.” as used herein (e.g., with reference to binding of a TMMP to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Affinity” refers to the strength of non-covalent binding. increased binding affinity being correlated with a lower Kp. “Specific binding” generally refers to binding of a ligand to a moiety that is its designated binding site or receptor. “Non-specific binding” generally refers to binding of a ligand to a moiety other than its designated binding site or receptor. Covalent binding” or “covalent bond,” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” and “patient,” are used interchangeably herein and refer to a human subject for whom diagnosis, treatment, or therapy is desired.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely does not induce cell lysis.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

Before the present disclosure is further described, it is to be understood that this invention is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to a “T-cell modulatory multimeric polypeptide” or TMMP includes a plurality of such polypeptides and reference to “the immunomodulatory polypeptide” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication. Further, the dates of publication provided may be different from the actual publication dates which may need to be independently confirmed.

### DETAILED DESCRIPTION

### T-Cell Modulatory Multimeric Polypeptides (TMMPS)

The present disclosure provides a TMMP comprising a heterodimeric polypeptide comprising: a) a first polypeptide comprising: i) a WT-1 peptide epitope; and ii) a first MHC polypeptide, which is a beta-2 microglobulin (β2M); b) a second polypeptide comprising i) a second MHC polypeptide, which is a class I MHC heavy chain polypeptide; ii) two variant IL-2 MODs in tandem, and iii) an immunoglobulin (Ig) Fc polypeptide.

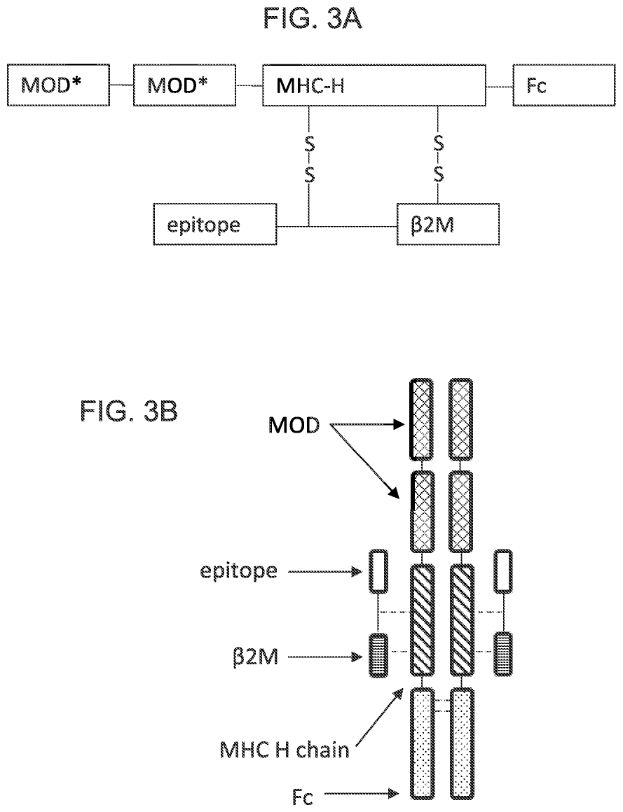

In an embodiment, the present disclosure provides a TMMP, wherein the TMMP is a heterodimer comprising: a) a first polypeptide comprising, in order from N-terminus to C-terminus: i) a WT-1 epitope; ii) a Cys-containing peptide linker;, and iii) a β2M polypeptide, and b) a second polypeptide comprising, in order from N-terminus to C-terminus: i) a first variant IL-2 polypeptide (MOD); ii) a peptide linker; iii) a second variant IL-2 polypeptide (MOD); iv) a peptide linker; v) a class I MHC heavy chain polypeptide; v) a peptide linker; and vi) an Ig Fc polypeptide. In an embodiment, the TMMP is a double disulfide-linked TMMP comprising polypeptides 1715Δ and 2380, as depicted in FIG. 3A.

In an embodiment, the present disclosure provides a TMMP, wherein the TMMP is a heterodimer comprising: a) a first polypeptide comprising, in order from N-terminus to C-terminus: i) a WT-1 epitope; ii) a Cys-containing peptide linker; and iii) a β2M polypeptide, and b) a second polypeptide comprising, in order from N-terminus to C-terminus: i) a class I MHC heavy chain polypeptide; ii) a peptide linker; iii) an Ig Fc polypeptide; iv) a peptide linker; v) a first variant IL-2 polypeptide (MOD); vi) a peptide linker; and vii) a second variant IL-2 polypeptide.

As discussed below, the variant IL-2 polypeptide MODs of the TMMPs exhibit reduced affinity for the co-MOD IL-2R. The combination of the reduced affinity of the variant IL-2 MOD for IL-2R, and the affinity of the WT-1 peptide epitope for a TCR, provides for enhanced selectivity of a TMMP. Thus, for example, a TMMP binds with higher avidity to a first T cell that displays both: i) a TCR specific for the epitope present in the TMMP; and ii) an IL-2R co-MOD, compared to the avidity to which it binds to a second T cell that displays: i) a TCR specific for an epitope other than the WT-1 epitope present in the TMMP; and ii) an IL-2R co-MOD. As a result, the TMMPs of this disclosure selectively bind to target T-cells having a TCR that specifically binds the WT-1 peptide epitope of the TMMP, compared to the binding of the TMMP to T cells that have a TCR that does not specifically bind the WT-1 peptide epitope of the TMMP. This selective binding means that the TMMPs will preferentially activate target T cells and will cause substantially less modulation/activation of non-target T cells, thereby reducing activation of non-target T cells and unwanted cytokine release.

### Dimerized TMMPs

A TMMP of the present disclosure can be dimerized; i.e., the present disclosure provides a TMMP comprising a dimer of a TMMP of the present disclosure. Thus, the present disclosure provides a TMMP comprising first and second heterodimers as described above, e.g., double disulfide-linked TMMPs comprising polypeptides 1715Δ and 2380, wherein the first heterodimer and the second heterodimer are covalently linked to one another through one or more (e.g., two) disulfide bonds that form spontaneously between the Ig Fc polypeptides in each heterodimer. See, e.g., FIG. 3B.

### MHC Polypeptides

As noted above, a TMMP of the present disclosure includes MHC polypeptides. For the purposes of this disclosure, the term “major histocompatibility complex (MHC) polypeptides” refers to human MHC polypeptides (also referred to as human leukocyte antigen (HLA)) polypeptides. The MHC polypeptides disclosed herein refer to class I MHC polypeptides (e.g., β2M and MHC class I heavy chain polypeptides). In this disclosure, both the β2M and MHC-H chain are of human origin, i.e., the MHC-H chain is an HLA heavy chain, or a variant thereof.

### MHC Heavy Chain

Unless expressly stated otherwise, a TMMP of the present disclosure does not include membrane anchoring domains (transmembrane regions) of an MHC class I heavy chain, or a part of MHC class I heavy chain sufficient to anchor the resulting TMMP to a cell (e.g., eukaryotic cell such as a mammalian cell) in which it is expressed. In some cases, the MHC class I heavy chain present in a TMMP of the present disclosure does not include a signal peptide, a transmembrane domain, or an intracellular domain (cytoplasmic tail) associated with a native MHC class I heavy chain. Thus, e.g., in some cases, the MHC class I heavy chain present in a TMMP of the present disclosure includes only the α1, α2, and α3 domains of an MHC class I heavy chain. In some cases, the MHC class I heavy chain present in a TMMP has a length of from about 270 amino acids (aa) to about 290 aa. In some cases, the MHC class I heavy chain present in a TMMP has a length of 270 aa, 271 aa, 272 aa, 273 aa, 274 aa, 275aa, 276 aa. 277 aa, 278 aa, 279 aa, 280 aa, 281 aa, 282 aa, 283 aa, 284 aa, 285 aa, 286 aa, 287 aa, 288 aa, 289 aa, or 290 aa.

Class I HLA heavy chain polypeptides include HLA-A heavy chain polypeptides, HLA-B heavy chain polypeptides, HLA-C heavy chain polypeptides. In some cases, a TMMP comprises a non-classical MHC Class I heavy chain polypeptide. Among the non-classical HLA heavy chain polypeptides, or portions thereof, that may be that may be incorporated into a TMMP of the present disclosure include, but are not limited to, those of HLA-E,-F, and-G alleles. Amino acid sequences for HLA-E,-F, and-G heavy chain polypeptides, (and the HLA-A, B and C alleles) may be found on the world wide web hla.alleles.org/nomenclature/index.html, the European Bioinformatics Institute (www (dot) ebi (dot) ac (dot) uk), which is part of the European Molecular Biology Laboratory (EMBL), and at the National Center for Biotechnology Information (www (dot) ncbi (dot) nlm (dot) nih (dot) gov).

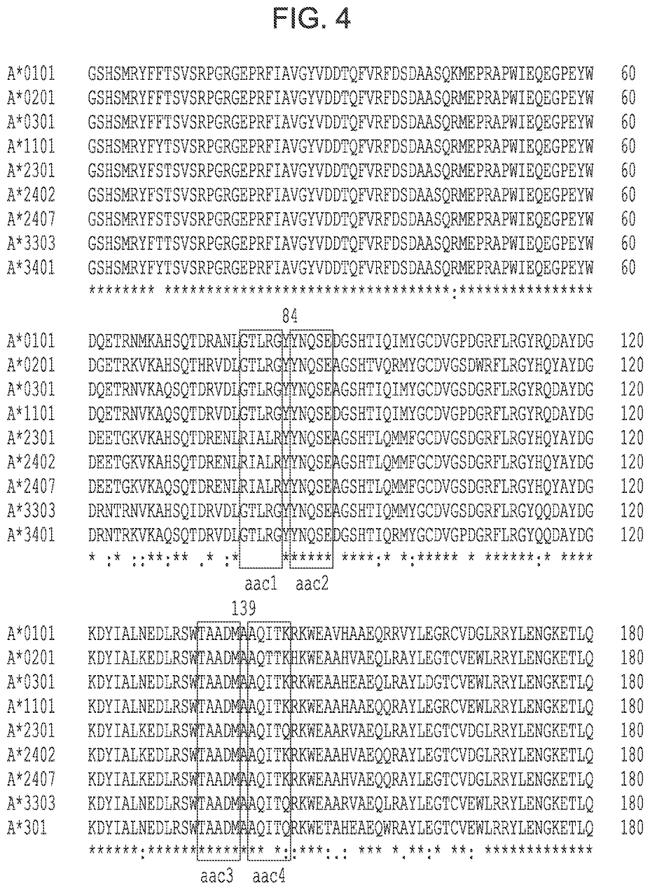

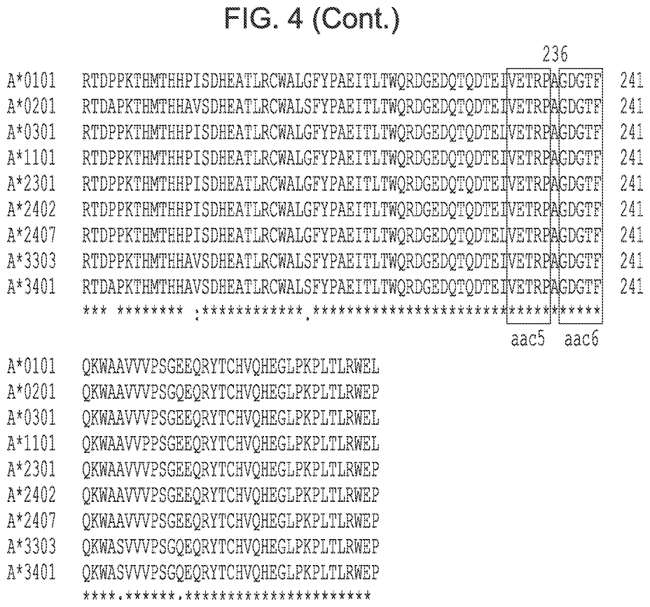

In some cases, a TMMP comprises an HLA-A heavy chain polypeptide. The HLA-A heavy chain peptide sequences, or portions thereof, that may be that may be incorporated into a TMMP of the present disclosure include, but are not limited to, the alleles: A*0101, A*0201, A*0301, A*1101, A*2301, A*2402, A*2407, A*3303, and A*3401, which are aligned without all, or substantially all, of the leader, transmembrane and cytoplasmic sequences in FIG. 4. Any of those alleles may comprise a mutation at one or more of positions 84, 139 and/or 236 (as shown in FIG. 4) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). See, e.g., published PCT Application WO 2020/132297 (Cue Biopharma, Inc.), published Jun. 25, 2020, for examples of specific HLA-A sequences. An HLA-A sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of one of the sequences of FIG. 4 may be employed.

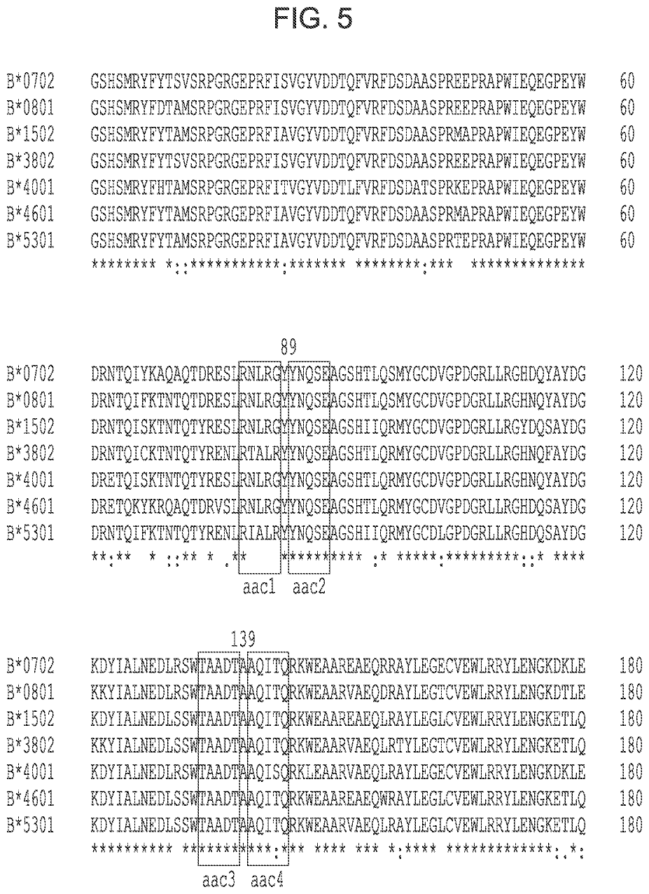

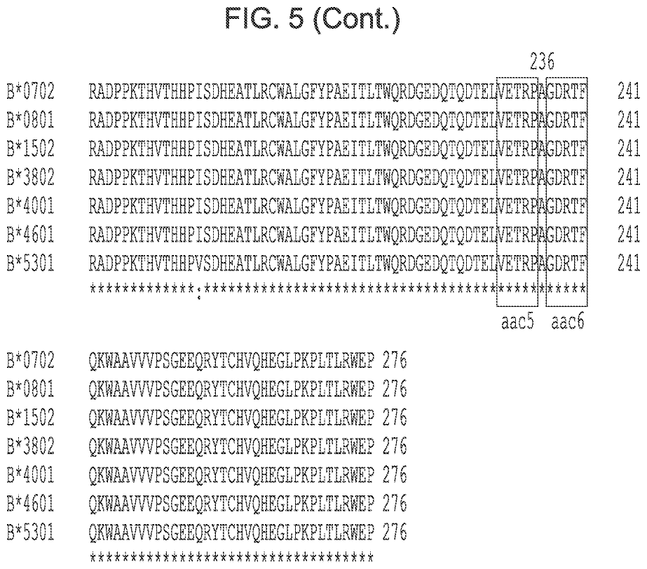

In some cases, a TMMP comprises an HLA-B heavy chain polypeptide. The HLA-B heavy chain peptide sequences, or portions thereof, that may be that may be incorporated into a TMMP include, but are not limited to, the alleles: B*0702, B*0801, B*1502, B*3802, B*4001, B*4601, and B*5301, which are aligned without all, or substantially all, of the leader, transmembrane and cytoplasmic sequences in FIG. 5. Any of those alleles may comprise a mutation at one or more of positions 84, 139 and/or 236 (as shown in FIG. 5) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). See, e.g., published PCT Application WO 2020/132297 (Cue Biopharma, Inc.), published June 25, 2020, for examples of specific HLA-B sequences. An HLA-B polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of one of the sequences of FIG. 5 may be employed.

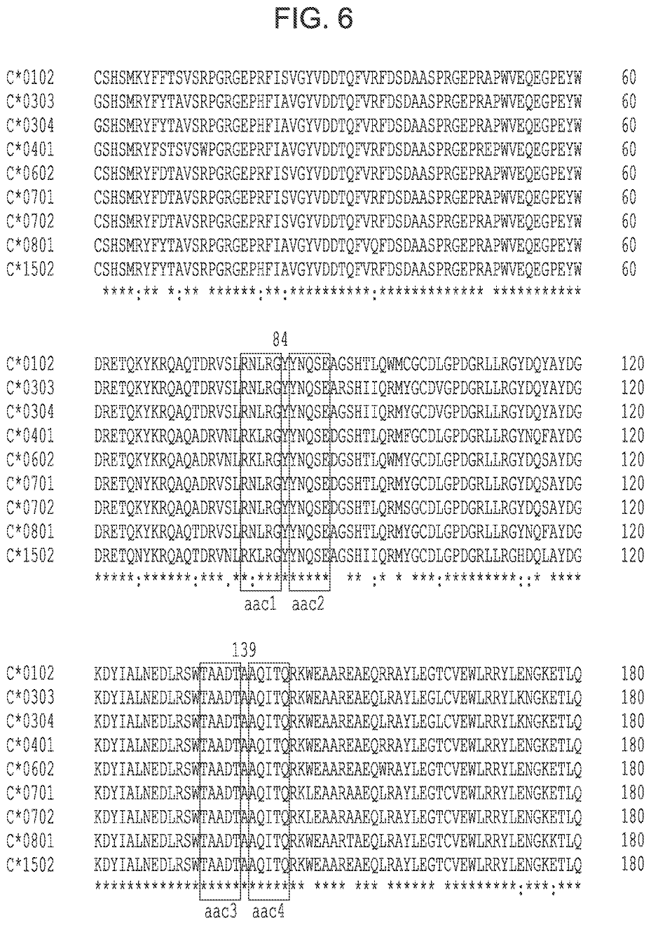

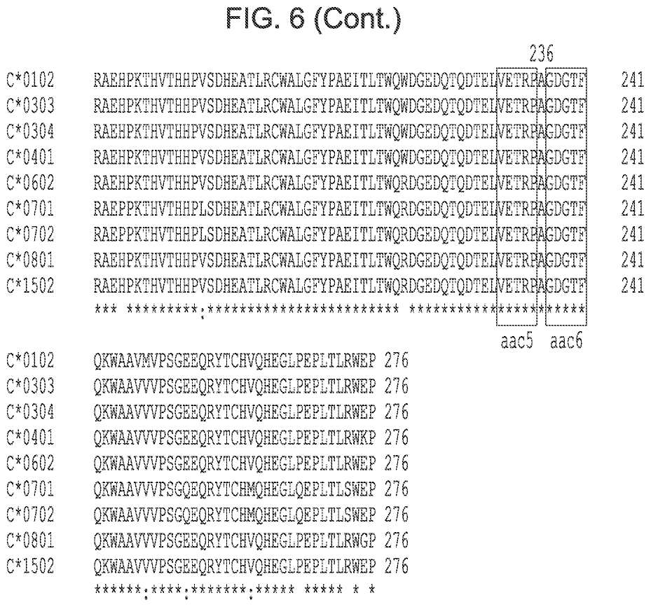

In some cases, a TMMP comprises an HLA-C heavy chain polypeptide. The HLA-C heavy chain polypeptide, or portions thereof, that may be that may be incorporated into a TMMP of the present disclosure include, but are not limited to, the alleles: C*0102, C*0303, C*0304, C*0401, C*0602. C*0701, C*0801, and C*1502, which are aligned without all, or substantially all, of the leader, transmembrane and cytoplasmic sequences in FIG. 6. Any of those alleles may comprise a mutation at one or more of positions 84, 139 and/or 236 (as shown in FIG. 6) selected from: a tyrosine to alanine substitution at position 84 (Y84A); a tyrosine to cysteine substitution at position 84 (Y84C); an alanine to cysteine substitution at position 139 (A139C); and an alanine to cysteine substitution at position 236 (A236C). See, e.g., published PCT Application WO 2020/132297 (Cue Biopharma, Inc.), published Jun. 25, 2020, for examples of specific HLA-C sequences. An HLA-C polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of one of the sequences of FIG. 6 may be employed.

Non-limiting examples of suitable HLA-E alleles include, but are not limited to, HLA-E*0101 (HLA-E*01:01:01:01), HLA-E*01: 03 (HLA-E*01:03:01:01), HLA-E*01:04, HLA-E*01:05, HLA-E*01:06, HLA-E*01:07, HLA-E*01:09, and HLA-E*01:10. Non-limiting examples of suitable HLA-F alleles include, but are not limited to, HLA-F*0101 (HLA-F*01:01:01:01), HLA-F*01:02, HLA-F*01:03 (HLA-F*01:03:01:01), HLA-F*01:04, HLA-F*01:05, and HLA-F*01:06. Non-limiting examples of suitable HLA-G alleles include, but are not limited to, HLA-G*0101 (HLA-G*01:01:01:01), HLA-G*01:02, HLA-G*01: 03 (HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA E, -F and -G alleles without all, or substantially all, of the leader, transmembrane and cytoplasmic sequences are provided in published PCT Application WO 2020/132297 (Cue Biopharma, Inc.), published Jun. 25, 2020.

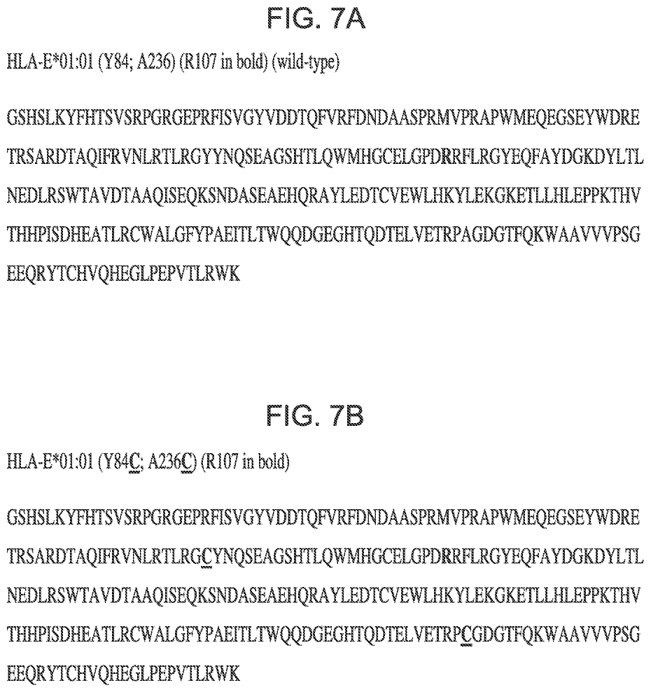

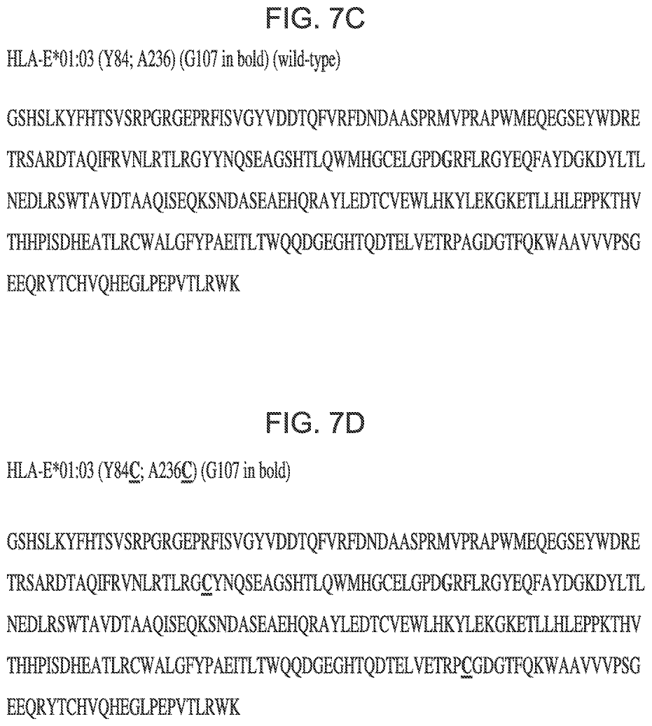

Amino acid sequences of HLA-E heavy chain polypeptides are provided in FIG. 7A-7D. where FIG. 7A provides the amino acid sequence of HLA-E*01:01 (wild-type); FIG. 7B provides the amino acid sequence of HLA-E*01:01 with Y84C and A2346C substitutions; FIG. 7C provides the amino acid sequence of HLA-E*01:03 (wild-type); and FIG. 7D provides the amino acid sequence of HLA-E*01:03 with Y84C and A2346C substitutions. An HLA-E sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequences of FIG. 7A-D may be employed.

In an embodiment, the MHC class I heavy chain is an HLA-A*02 heavy chain polypeptide. In an embodiment, e.g., double disulfide-linked TMMPs comprising polypeptides 1715Δ and 2380, or a dimer thereof. In some cases, an MHC class I heavy chain polypeptide present in a TMMP comprises an amino acid sequence having at least 75%, at least 80%, at least 85%, at least 90%, at least 95%, at least 98%, at least 99%, or 100%, amino acid sequence identity to the HLA heavy chain set forth in the following amino acid sequence: GSHSMRYFFTSVSRPGRGEPRFIAVGYVDDTQFVRFDSDAASQRMEPRAPWIEQEGPEYWDGET RKVKAHSQTHRVDLGTLRGYYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPAGDGTFQKWAAVV VPSGQEQRYTCHVQHEGLPKPLTLRWEP (SEQ ID NO:1). This HLA-A heavy chain polypeptide is also referred to as “HLA-A*0201” or simply “HLA-A02.” In some cases, the C-terminal Pro is not included in a TMMP. For example, in some cases, an HLA-A02 polypeptide suitable for inclusion in a TMMP comprises a polypeptide having at least 75%, at least 80%, at least 85%, at least 90%, at least 95%, at least 98%, at least 99%, or 100%, amino acid sequence identity to following amino acid sequence:

HLA-A (Y84C; A236C)
(SEQ ID NO: 2)
GSHSMRYFFTSVSRPGRGEPRFIAVGYVDDTQFVRFDSDAASQRMEPRA
PWIEQEGPEYWDGETRKVKAHSQTHRVDLGTLRGYYNQSEAGSHTVQRM
YGCDVGSDWRFLRGYHQYAYDGKDYIALKEDLRSWTAADMAAQTTKHKW
EAAHVAEQLRAYLEGTCVEWLRRYLENGKETLQRTDAPKTHMTHHAVSD
HEATLRCWALSFYPAEITLTWQRDGEDQTQDTELVETRPAGDGTFQKWA
AVVVPSGQEQRYTCHVQHEGLPKPLTLRWE.

In some cases, an HLA-A heavy chain polypeptide suitable for inclusion in a TMMP comprises an amino acid sequence having at least 75%, at least 80%, at least 85%, at least 90%, at least 95%, at least 98%, at least 99%, or 100%, amino acid sequence identity to the following human HLA-A heavy chain (Y84C; A236C) amino acid sequence: GSHSMRYFFTSVSRPGRGEPRFIAVGYVDDTQFVRFDSDAASQRMEPRAPWIEQEGPEYWDGET RKVKAHSQTHRVDLGTLRGCYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPCGDGTFQKWAAVV VPSGQEQRYTCHVQHEGLPKPLTLRWE (SEQ ID NO:3), where amino acid 84 is a Cys and where amino acid 236 is a Cys.

### Beta-2 Microglobulin

A β2-microglobulin (β2M) polypeptide of a TMMP of the present disclosure is a human β2M polypeptide having at least 75%, at least 80%, at least 85%, at least 90%, at least 95%, at least 98%, at least 99%, or 100%, amino acid sequence identity to the following amino acid sequence:

(SEQ ID NO: 4)
IQRTPKIQVY SCHPAENGKS NFLNCYVSGF
HPSDIEVDLLKNGERIEKVE
HSDLSFSKDW SFYLLYYTEF TPTEKDEYAC RVNHVTLSQP
KIVKWDRDM

### Multiple Disulfide Bonded TMMPs

In the embodiments herein, the first polypeptide and the second polypeptide of a TMMP of the present disclosure are linked to one another by at least two disulfide bonds (i.e., two interchain disulfide bonds). At least one of the at least two disulfide bonds links a Cys in the linker to a Cys in an MHC class I heavy chain polypeptide, and at least one of the two disulfide bonds links a Cys in β2M polypeptide to a Cys in an MHC class I heavy chain. Examples of such multiple disulfide-linked TMMP are depicted schematically in FIG. 3A (heterodimer) and FIG. 3B (dimer).

In some cases, a multiple disulfide-linked TMMP (e.g., a double disulfide-linked TMMP) exhibits increased stability, compared to a control TMMP that includes only one of the at least two disulfide bonds. In some cases, a multiple disulfide-linked TMMP (e.g., a double disulfide-linked TMMP) exhibits increased in vitro stability, compared to a control TMMP that includes only one of the at least two disulfide bonds.

Whether a multiple disulfide-linked TMMP (e.g., a double disulfide-linked TMMP) exhibits increased in vitro stability, compared to a control TMMP that includes only one of the at least two disulfide bonds, can be determined by measuring the amount disulfide-linked heterodimeric TMMP present in a sample over time and/or under a specified condition and/or during purification of the TMMP.

As an example, a double disulfide-linked TMMP comprising polypeptides 1715Δ and 2380 exhibits greater in vitro stability, compared to a TMMP comprising similar polypeptides comprising only one of the above-described disulfide bonds

In some cases, a multiple disulfide-linked TMMP exhibits increased in vivo stability, compared to a similar TMMP that includes only one of the at least two disulfide bonds.

In some cases, the presence of the two disulfide bonds provides for increased production of the TMMP, compared to a similar TMMP that includes only one of the at least two disulfide bonds, e.g., when produced in a mammalian cell in in vitro cell culture.

### Ig Fc polypeptides

As noted above, a TMMP comprise an Fc polypeptide. The Fc polypeptide of a TMMP can be a human IgG1 Fc comprising an amino acid sequence having at least about 70%, at least about 75%, at least about 80%, at least about 85%, at least about 90%, at least about 95%, at least about 98%, at least about 99%, or 100%, amino acid sequence identity to one of the following amino acid sequences:

DKTHTCPPCPAPELLGGPSVFLFPPKPKDTLMISRTPEVTCVVVDVSHEDPEVKEN WYVDGVEVHNAKTKPREEQYNSTYRVVSVLTVLHQDWLNGKEYKCKVSNKALPAPIEKTISK AKGQPREPQVYTLPPSREEMTKNQVSLTCLVKGFYPSDIAVEWESNGQPENNYKTTPPVLDSDG SFFLYSKLTVDKSRWQQGNVFSCSVMHEALHNHYTQKSLSLSPG (SEQ ID NO:5); and has a length of 226 amino acids, or DKTHTCPPCPAPELLGGPSVFLFPPKPKDTLMISRTPEVTCVVVDVSHEDPEVKEN WYVDGVEVHNAKTKPREEQYNSTYRVVSVLTVLHQDWLNGKEYKCKVSNKALPAPIEKTISK AKGQPREPQVYTLPPSREEMTKNQVSLTCLVKGFYPSDIAVEWESNGQPENNYKTTPPVLDSDG SFFLYSKLTVDKSRWQQGNVFSCSVMHEALHNHYTQKSLSLSPGK (SEQ ID NO:6); and has a length of 227 amino acids. This sequence is identical to the above sequence, but includes a C-terminal lysinc.

In some cases, the Fc polypeptide present in a TMMP is a variant of a human IgG1 Fc polypeptide, which variant has a substantially reduced ability to effect complement-dependent cytotoxicity (CDC) and/or antibody-dependent cell cytotoxicity (ADCC). In some cases, the Fc polypeptide present in a TMMP comprises an N297A substitution, which is N77 of the amino acid sequences depicted above. In some cases, the Fc polypeptide present in a TMMP comprises a substitution of L234 (L14 of the IgG1 Fc amino acid sequences depicted above) with an amino acid other than leucine, e.g., alanine (a L234A substitution or an L14A substitution of the IgG1 Fc amino acid sequences depicted above). In some cases, the Fc polypeptide present in a TMMP comprises a substitution of L235 (L15 of the IgG1 Fc amino acid sequences depicted above) with an amino acid other than leucine, e.g., alanine (an L235A substitution or an L15A substitution of the IgG1 Fc amino acid sequences depicted above). In some cases, e.g., for polypeptide 17154, the Fc polypeptide present in a TMMP comprises both a substitution of L234 (L14 of the IgG1 Fc amino acid sequences depicted above) with an amino acid other than leucine, e.g., alanine, and a substitution of L235 (L15 of the IgG1 Fc amino acid sequences depicted above) with an amino acid other than leucine, e.g., alanine (an L235A substitution or an L15A substitution in either of the two IgG1 Fc amino acid sequences depicted above). Accordingly, in some cases, the IgG1 Fc comprises an amino acid sequence having at least about 70%, at least about 75%, at least about 80%, at least about 85%, at least about 90%, at least about 95%, at least about 98%, at least about 99%, or 100%, amino acid sequence identity to one of the following amino acid sequences:

DKTHTCPPCPAPEAALLGGPSVFLFPPKPKDTLMISRTPEVTCVVVDVSHEDPEVK FNWYVDGVEVHNAKTKPREEQYNSTYRVVSVLTVLHQDWLNGKEYKCKVSNKALPAPIEKTI SKAKGQPREPQVYTLPPSREEMTKNQVSLTCLVKGFYPSDIAVEWESNGQPENNYKTTPPVLDS DGSFFLYSKLTVDKSRWQQGNVFSCSVMHEALHNHYTQKSLSLSPG (SEQ ID NO:7); and has a length of 226 amino acids and is the IgG1 Fc polypeptide of 1715Δ, or DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO:8); and has a length of 227 amino acids. This sequence is identical to the above sequence, but includes a C-terminal lysine.

### Linkers

As noted above, the peptide linker joining the WT-1 peptide epitope comprises a Cys residue. In some cases, the peptide linker comprises the amino acid sequence GCGGS (SEQ ID NO:9). In some cases, the peptide linker comprises the amino acid sequence GCGGS(GGGGS)n (SEQ ID NO: 56), where n is an integer from 1 to 10. In some cases, the peptide linker comprises the amino acid sequence GCGGS(GGGGS)n (SEQ ID NO:10), where n is 1. In some cases, the peptide linker comprises the amino acid sequence GCGGS(GGGGS)n (SEQ ID NO:11), where n is 2. In some cases, the peptide linker comprises the amino acid sequence GCGGS(GGGGS)n (SEQ ID NO:12), where n is 3. In some cases, the peptide linker comprises the amino acid sequence GCGGS(GGGGS)n (SEQ ID NO:13), where n is 4. In some cases, the peptide linker comprises the amino acid sequence GCGGS(GGGGS)n (SEQ ID NO:14), where n is 5. In some cases, the peptide linker comprises the amino acid sequence GCGGS(GGGGS)n (SEQ IDNO: 15), where n is 6. In some cases, the peptide linker comprises the amino acid sequence GCGGS(GGGGS)n (SEQ ID NO:16), where n is 7. In some cases, the peptide linker comprises the amino acid sequence GCGGS(GGGGS)n (SEQ ID NO:17), where n is 8. In some cases, the peptide linker comprises the amino acid sequence GCGGS(GGGGS)n (SEQ ID NO:18), where n is 9. In some cases, the peptide linker comprises the amino acid sequence GCGGS(GGGGS)n (SEQ ID NO: 19), where n is 10.

In some cases, the peptide linker comprises the amino acid sequence CGGGS (SEQ ID NO: 20). In some cases, the peptide linker comprises the amino acid sequence CGGGS(GGGGS)n (SEQ ID NO: 21), where n is an integer from 1 to 10. In some cases, the peptide linker comprises the amino acid sequence CGGGS(GGGGS)n (SEQ ID NO:22), where n is 1. In some cases, the peptide linker comprises the amino acid sequence CGGGS(GGGGS)n (SEQ ID NO:23), where n is 2. In some cases, the peptide linker comprises the amino acid sequence CGGGS(GGGGS)n (SEQ ID NO:24), where n is 3. In some cases, the peptide linker comprises the amino acid sequence CGGGS(GGGGS)n (SEQ ID NO: 25), where n is 4. In some cases, the peptide linker comprises the amino acid sequence CGGGS(GGGGS)n (SEQ ID NO:26), where n is 5. In some cases, the peptide linker comprises the amino acid sequence CGGGS(GGGGS)n (SEQ ID NO:27), where n is 6. In some cases, the peptide linker comprises the amino acid sequence CGGGS(GGGGS)n (SEQ ID NO:28), where n is 7. In some cases, the peptide linker comprises the amino acid sequence CGGGS(GGGGS) n (SEQ ID NO:29), where n is 8. In some cases, the peptide linker comprises the amino acid sequence CGGGS(GGGGS) n (SEQ ID NO:30), where n is 9. In some cases, the peptide linker comprises the amino acid sequence CGGGS(GGGGS)n (SEQ ID NO:31), where n is 10.

A TMMP of the present disclosure also includes one or more other independently selected linkers that do not comprise a Cys, i.e., between one or more of: i) an MHC class I polypeptide and an Ig Fc polypeptide; ii) a MOD and an MHC class I polypeptide; iii) a first MOD and a second MOD. Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

Exemplary linkers include glycine polymers (G)ₙ, glycine-serine polymers (including, for example. (GS)ₙ. (GSGGS)ₙ, (SEQ ID NO:32) and (GGGS), (SEQ ID NO:33), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:34), GGSGG (SEQ ID NO:35), GSGSG (SEQ ID NO:36), GSGGG (SEQ ID NO:37), GGGSG (SEQ ID NO:38), GSSSG (SEQ ID NO:39), and the like. Exemplary linkers can include, e.g., Gly(Sera)n (SEQ ID NO:40), where n is 1, 2, 3, 4, 5, 6, 7, 8, 9, or 10, e.g., amino acid sequence (GGGGS)n (SEQ ID NO:41), where n is 1, amino acid sequence (GGGGS)n (SEQ ID NO:42), where n is 2, amino acid sequence (GGGGS)n (SEQ ID NO:43), where n is 3, amino acid sequence (GGGGS)n (SEQ ID NO:44), where n is 4, amino acid sequence (GGGGS)n (SEQ ID NO:45), where n is 5, amino acid sequence (GGGGS)n (SEQ ID NO: 46), where n is 6, amino acid sequence (GGGGS)n (SEQ ID NO:47), where n is 7, amino acid sequence (GGGGS)n (SEQ ID NO:48), where n is 8, amino acid sequence (GGGGS)n (SEQ ID NO:49), where n is 9, and amino acid sequence (GGGGS)n (SEQ ID NO:89), where n is 10. In some cases, a linker comprises the amino acid sequence AAAGG (SEQ ID NO:51). In some cases, a linker comprises the amino acid sequence (GSSSS)n (SEQ ID NO: 52), where n is 4. In some cases, a linker comprises the amino acid sequence (GSSSS)n (SEQ ID NO:53), where n is 5.

### Epitopes

The epitope present in a TMMP is a WT-1 peptide, e.g., a WT-1 peptide that, together with MHC, presents an epitope to a TCR. Amino acid sequences of WT-1 isoforms are disclosed in WO 2020/132297 (Cue Biopharma, Inc.), published Jun. 25, 2020. In some cases, the WT-1 peptide present in a TMMP presents an HLA-A*0201-restricted epitope. WT-1 peptides that present an HLA-A*0201-restricted epitope include, e.g., VLDFAPPGA (SEQ ID NO:60) (WT-1 37-45).

### HLA/Peptide Binding Assays

Whether a given peptide (e.g., WT-1 peptide) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays. See, e.g., published PCT Application WO 2020/132297 (Cue Biopharma, Inc.), published Jun. 25, 2020.

### Immunomodulatory Polypeptides (“MODs”)

For purposes of this disclosure, the MOD and its cognate co-MOD are IL-2 and IL-2 receptor (IL-2R). Wild-type IL-2 binds to IL-2 receptor (IL-2R), i.e., a heterotrimeric polypeptide comprising IL-2Rα, IL-2Rβ, and IL-2Rγ.

An IL-2 receptor is in some cases a heterotrimeric polypeptide comprising an alpha chain (IL-2Rα; also referred to as CD25), a beta chain (IL-2Rβ; also referred to as CD122: and a gamma chain (IL-2Rγ; also referred to as CD132). Amino acid sequences of human IL-2, human IL-2Rα, IL-2Rβ, and IL-2Rγ are known. See, e.g., published PCT applications WO2020132138A1, WO2019/051091 and WO 2020/132297.

The IL-2 MOD present in a TMMP of the present disclosure is a variant IL-2 polypeptide that exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2Rβ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ such that the IL-2 variant polypeptide exhibits an overall reduced affinity for IL-2R. Such variants are disclosed in published PCT applications WO2020132138A1, WO2019/051091 and WO 2020/132297. Such variants also may exhibit decreased binding to IL-2Rγ such that the IL-2 variant polypeptide exhibits an overall reduced affinity for IL-2R.

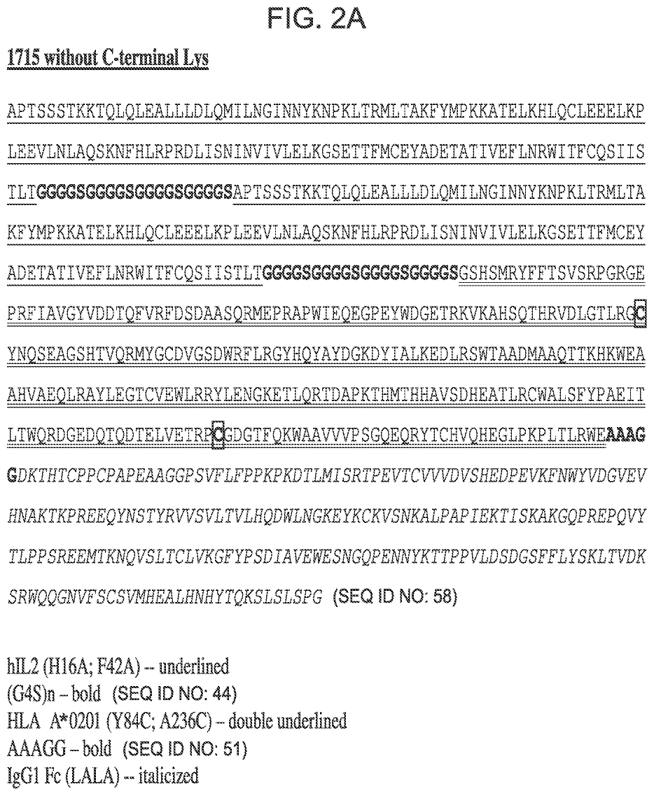

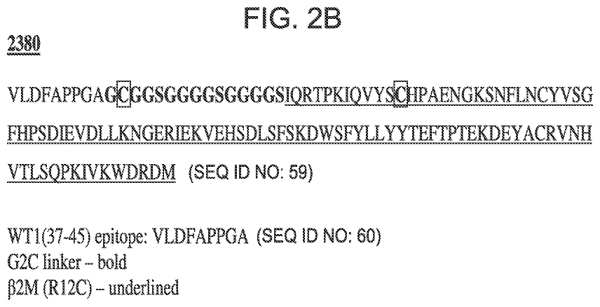

IL-2 variant MODs that exhibit decreased or substantially no binding to IL-2Rα, and also exhibit decreased binding to IL-2Rβ such that the IL-2 variant polypeptide exhibits an overall reduced affinity for IL-2R are disclosed in published PCT applications WO2020132138A1, WO2019/051091 and WO 2020/132297. For example, IL-2 variants having substitutions at H16 and F42 (e.g., the IL-2 variants in the 1715Δ polypeptide, shown in FIG. 2A, each of which have H16A and F42A substitutions) have shown decreased binding to IL-2Ra and IL-2RB. See, Quayle et al., Clin Cancer Res; 26 (8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type IL2 binding, predominantly due to a faster off-rate for each of these interactions. TMPs comprising such variants, including variants that exhibit decreased binding to IL-2Rα and IL-2Rβ, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically binds the peptide epitope on the TMP. That is, the binding of the IL-2 variant MOD to its costimulatory polypeptide on the T cell is substantially driven by the binding of the MHC-epitope moiety rather than by the binding of the IL-2.

Suitable IL-2 variant MODs thus include a polypeptide that comprises an amino acid sequence having at least 90%, at least 95%, at least 98%, or at least 99% amino acid sequence identity to the wild-type IL-2 amino acid sequence: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:54), and that have one or more amino acid differences from the wild-type IL-2 amino acid sequence that cause the variant to exhibit decreased or substantially no binding to IL-2Rα, and also decreased binding to IL-2Rβ.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:55), i.e., the variant IL-2 polypeptide has the amino acid sequence of wild-type IL-2 but with H16A and F42A substitutions (shown in bold). Alternatively, the foregoing sequence, but with substitutions other than Ala at H16 and/or F42 may be employed, e.g., H16T may be employed instead of H16A. In embodiments, a TMP comprises two copies of such a variant IL-2 polypeptide in tandem, where such IL-2 variants are linked together by a peptide linker. See, e.g., FIG. 2A.

### Exemplary TMMPs

The present disclosure provides a TMMP comprising a heterodimeric polypeptide comprising: a) a first polypeptide comprising: i) a WT-1 peptide epitope; and ii) a first MHC polypeptide, which is a β2M; b) a second polypeptide comprising i) a second MHC polypeptide, which is a class I MHC heavy chain polypeptide; ii) two variant IL-2 MODs in tandem, and iii) an immunoglobulin (Ig) Fc polypeptide.

In an embodiment, the present disclosure provides a TMMP, wherein the TMMP is a heterodimer comprising: a) a first polypeptide comprising, in order from N-terminus to C-terminus: i) a WT-1 epitope, ii) a Cys-containing peptide linker, and iii) a β2M polypeptide; and b) a second polypeptide comprising, in order from N-terminus to C-terminus: i) a first variant IL-2 polypeptide (MOD), ii) a peptide linker, iii) a second variant IL-2 polypeptide (MOD), iv) a peptide linker, v) a class I MHC heavy chain polypeptide, v) a peptide linker; and vi) an IgG1 Fc polypeptide. In an embodiment, the TMMP is a double disulfide-linked TMMP comprising polypeptides 1715Δ and 2380, as depicted in FIG. 3A. In a further embodiment, the TMMP is a homodimer comprising two TMMPs comprising polypeptides 1715Δ and 2380, as depicted in FIG. 3B.

In an embodiment, the present disclosure provides a TMMP, wherein the TMMP is a heterodimer comprising: a) a first polypeptide comprising, in order from N-terminus to C-terminus: i) a WT-1 epitope; ii) a Cys-containing peptide linker; and iii) a β2M polypeptide, and b) a second polypeptide comprising, in order from N-terminus to C-terminus: i) a class I MHC heavy chain polypeptide; ii) a peptide linker; iii) an Ig Fc polypeptide; iv) a peptide linker; v) a first variant IL-2 polypeptide (MOD); vi) a peptide linker; and vii) a second variant IL-2 polypeptide.

In some cases, the Ig Fc polypeptide is a human IgG1 Fc polypeptide In some cases, the Ig Fc polypeptide is a variant of a human IgG1 Fc polypeptide, which variant has a substantially reduced ability to effect complement-dependent cytotoxicity (CDC) or antibody-dependent cell cytotoxicity (ADCC), e.g., by providing L234A and L235A substitutions. In some cases, the MOD is a variant IL-2 polypeptide comprising H16A and F42A substitutions. In some cases, the MOD is a variant IL-2 polypeptide comprising H16T and F42A substitutions.

In some cases, the Cys-containing peptide linker comprises the amino acid sequence GCGGS(GGGGS)n (SEQ ID NO:57), where n is an integer from 1 to 9, e.g., where n is 2, 3, or 4 such as GCGGS(GGGGS)n (SEQ ID NO:10), where n is 2. In some cases, a peptide linker that does not comprise a Cys is between one or more of: i) the MHC H chain polypeptide and the IgG1 Fc polypeptide; ii) the first MHC polypeptide and the MOD; and iii) between the two MODs. In some cases, such peptide linker comprises the amino acid sequence AAAGG (SEQ ID NO:51). In some cases, such peptide linker comprises the amino acid sequence (GGGGS)n (SEQ ID NO:50), where n is an integer from 1 to 10, e.g., where n is 2, 3, or 4 such as (GGGGS)n (SEQ ID NO:44), where n is 4. The peptide linkers are independently selected, i.e., they may be the same or different.

### Methods of Generating a Multimeric T-Cell Modulatory Polypeptide

Methods of producing a TMMP as disclosed herein are described in WO 2020/132297 (Cue Biopharma, Inc.), published Jun. 25, 2020.

### Compositions

The present disclosure provides compositions, including pharmaceutical compositions, comprising a TMMP (synTac) of the present disclosure. The present disclosure provides compositions, including pharmaceutical compositions, comprising a TMMP.

A wide variety of pharmaceutically acceptable ingredients are known in the art and hence are not discussed in detail herein. Moreover, pharmaceutically acceptable ingredients and compositions have been amply described in a variety of publications, including, but not limited to, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3rd ed. Amer. Pharmaceutical Assoc. Many other publications describing preparation of biopharmaceutical compositions may be consulted.

The composition may be formulated according to the various routes of administration described below. Generally speaking, pharmaceutical compositions comprising the TMMPs of this disclosure, e.g., a homodimer comprising two 17154-2380 TMMPs, will be an aqueous liquid and typically will be administered via an intravenous infusion. In some cases, the pharmaceutical composition comprising the TMMP can be admixed with saline (e.g., 0.9% NaCl) prior to IV administration. Thus, the present disclosure provides a sterile composition comprising: a) a TMMP of the present disclosure; and b) saline (e.g., 0.9% NaCl). Alternatively, it may be administered neat via an intravenous infusion, i.e., without further dilution. Alternatively, the pharmaceutical composition may be formulated so as to be administered by injection.

Conventional and pharmaceutically acceptable routes of administration include intratumoral, peritumoral, intramuscular, intralymphatic, intratracheal, intracranial, intrathecal, subcutaneous, intradermal, topical application, intravenous, intraarterial, rectal, nasal, oral, and other enteral and parenteral routes of administration. As noted above, a pharmaceutical composition comprising a TMMP typically will be administered intravenously, but may also be administered by other routes that involve injection.

### Methods of Modulating T Cell Activity

The present disclosure provides a method of selectively modulating the activity of an epitope-specific T cell, the method comprising contacting the T cell with a TMMP of the present disclosure, where contacting the T cell with a TMMP selectively modulates the activity of the epitope-specific T cell. In some cases, the contacting occurs in vitro. In some cases, the contacting occurs in vivo. In some cases, the contacting occurs ex vivo. In some instances, the TMMP increases cytotoxic activity of the T cell toward the cancer cell and/or the number of the epitope-specific T cells.

The present disclosure provides a method of detecting, in a mixed population of T cells obtained from an individual, the presence of a target T cell that binds an epitope of interest (e.g., a WT-1 epitope), the method comprising: a) contacting in vitro the mixed population of T cells with a TMMP, wherein the TMMP comprises the WT-1 epitope; and b) detecting activation and/or proliferation of T cells in response to said contacting, wherein activated and/or proliferated T cells indicates the presence of the target T cell.

### Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A TMMP of the present disclosure may be administered in amounts between 1 ng/kg body weight and 20 mg/kg body weight per dose, e.g. from 0.1 mg/kg body weight to 10 mg/kg body weight, e.g. from 1 mg/kg body weight to 5 mg/kg or from 5 mg/kg body weight to 10 mg/kg body weight; however, doses below or above this exemplary range are envisioned, especially considering the aforementioned factors. If the regimen is a continuous infusion, it can also be in the range of 1 ug to 10 mg per kilogram of body weight per minute. Generally speaking, a TMMP of the present disclosure can be administered in an amount of from about 1 mg/kg body weight to 20 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, or from about 15 mg/kg body weight to about 20 mg/kg body weight. Typical ranges may be from 1 mg/kg body weight to 5 mg/kg body weight or from 5 mg/kg body weight to about 10 mg/kg body weight, e.g., 1, 2, 4, 5, 6, 7 or 8 mg/kg body weight

Following successful treatment, it may be desirable to have the patient undergo maintenance therapy to prevent the recurrence of the disease state, wherein a TMMP is administered in maintenance doses in the above ranges.

Those of skill will readily appreciate that dose levels can vary as a function of the specific TMMP, the severity of the symptoms and the susceptibility of the subject to side effects. Preferred dosages for a given compound are readily determinable by those of skill in the art by a variety of means.

The frequency of administration of a TMMP can vary depending on any of a variety of factors, but generally speaking will be administered once a week, once every two weeks, once every three weeks, once every four weeks, once per month, or less frequently than once per month, e.g., once every five weeks, once every six weeks, once every two months, once every three months, etc., but also can be administered more frequently than once per week, e.g., twice per week (biw), three times per week (tiw), four times per week, five times per week, six times per week, every other day (qod), or daily (qd). In some cases, the TMMP is administered once every three weeks. Administration Administration generally should be stopped upon disease progression or unacceptable toxicity.

The duration of administration of a TMMP can vary, depending on any of a variety of factors, e.g., patient response, etc. For example, a TMMP can be administered over a period of time ranging from one month to about two months, from about two months to about four months, from about four months to about six months, from about six months to about eight months, from about eight months to about 1 year, from about 1 year to about 2 years, or from about 2 years to about 4 years, or more. Typically, the TMMP will continue to be dosed for at least as long as the patient continues to receive a clinically determined benefit, which likely will be from at least many months to multiple years.

### Combination Therapies

In some cases, a method of the present disclosure for treating cancer in an individual comprises: a) administering a TMMP; and b) administering at least one additional therapeutic agent or therapeutic treatment. Suitable additional therapeutic agents include, but are not limited to, a small molecule cancer chemotherapeutic agent, and an immune checkpoint inhibitor. Suitable additional therapeutic treatments include, e.g., radiation, surgery (e.g., surgical resection of a tumor), and the like.

A treatment method of the present disclosure can comprise co-administration of a TMMP and at least one additional therapeutic agent. By “co-administration” is meant that both a TMMP and at least one additional therapeutic agent are administered to an individual, although not necessarily at the same time, in order to achieve a therapeutic effect that is the result of having administered both the TMMP and the at least one additional therapeutic agent. The administration of the TMMP and the at least one additional therapeutic agent can be substantially simultaneous, e.g., the TMMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a TMMP of the present disclosure is administered to an individual who is undergoing treatment with, or who has undergone treatment with, the at least one additional therapeutic agent. The administration of the TMMP can occur at different times and/or at different frequencies.

As an example, a treatment method of the present disclosure can comprise co-administration of a TMMP and an immune checkpoint inhibitor such as an antibody specific for an immune checkpoint. By “co-administration” is meant that both a TMMP and an immune checkpoint inhibitor (e.g., an antibody specific for an immune checkpoint polypeptide) are administered to an individual, although not necessarily at the same time, in order to achieve a therapeutic effect that is the result of having administered both the TMMP and the immune checkpoint inhibitor (e.g., an antibody specific for an immune checkpoint polypeptide). The administration of the TMMP and the immune checkpoint inhibitor (e.g., an antibody specific for an immune checkpoint polypeptide) can be substantially simultaneous, e.g., the TMMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the immune checkpoint inhibitor (e.g., an antibody specific for an immune checkpoint polypeptide). In some cases, a TMMP of the present disclosure is administered to an individual who is undergoing treatment with, or who has undergone treatment with, an immune checkpoint inhibitor (e.g., an antibody specific for an immune checkpoint polypeptide). The administration of the TMMP and the immune checkpoint inhibitor (e.g., an antibody specific for an immune checkpoint polypeptide) can occur at different times and/or at different frequencies. Where there is an established dosing interval for the checkpoint inhibitor, depending on the interval, it may be possible to administer the TMMP on the same day as the checkpoint inhibitor. For example, in some cases, where the dosing schedule for pembrolizumab is once every three weeks, the pharmaceutical composition comprising the TMMP may be administered on the same day.

Exemplary immune checkpoint inhibitors include inhibitors that target an immune checkpoint polypeptide such as CD27, CD28, CD40, CD122, CD96, CD73, CD47, OX40, GITR, CSFIR, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT and VISTA.

In some cases, the immune checkpoint inhibitor is an antibody specific for an immune checkpoint polypeptide. In some cases, the anti-immune checkpoint antibody is a monoclonal antibody. In some cases, the anti-immune checkpoint antibody is humanized, or de-immunized such that the antibody does not substantially elicit an immune response in a human. In some cases, the anti-immune checkpoint antibody is a humanized monoclonal antibody. In some cases, the anti-immune checkpoint antibody is a de-immunized monoclonal antibody. In some cases, the anti-immune checkpoint antibody is a fully human monoclonal antibody. In some cases, the anti-immune checkpoint antibody inhibits binding of the immune checkpoint polypeptide to a ligand for the immune checkpoint polypeptide. In some cases, the anti-immune checkpoint antibody inhibits binding of the immune checkpoint polypeptide to a receptor for the immune checkpoint polypeptide.

Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), also known as BMS-936559, CK-301, CS-1001, SHR-1316, CBT-502 (also known as TQB-2450), BGB-A333, MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CellDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035 (also known as Envafolimab), cemiplimab (Libtayo®); and combinations thereof. For example, in some cases, the immune checkpoint inhibitor is an anti-PD-1 antibody. Suitable anti-PD-1 antibodies include, e.g., nivolumab, pembrolizumab (also known as MK-3475), cemiplimab, pidilizumab, SHR-1210, PDR001, and AMP-224. In some cases, the anti-PD-1 monoclonal antibody is nivolumab, pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MEDI4736, MPDL3280A (also known as RG7446), KN035, or MSB0010718C. In some embodiments, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149. In some cases, the immune checkpoint inhibitor is an anti-TIGIT antibody that binds to T-cell immunoreceptor with immunoglobulin and ITIM domains (TIGIT). In some cases, the anti-TIGIT antibody is BMS-986207 (Bristol-Myers Squibb). In some cases, the anti-TIGIT antibody is tiragolumab. In some cases, the anti-TIGIT antibody is EOS88448 (EOS-448). See, e.g., U.S. Pat. No. 11,008,390 and U.S. Pat. No. 10,189,902; U.S. Patent Publication No. 2017/0088613; and WO 2019/137541.

### Treatment Methods

The present disclosure provides a method of treatment of an individual, the method comprising administering to the individual an amount of a TMMP of the present disclosure. The TMMP comprising a WT-1 peptide epitope can be administered to an individual having a WT-1-expressing cancer. WT1-expressing cancers include solid-tumor cancers and hematologic cancers. Such cancers can include, e.g., a leukemia, a desmoplastic small round cell tumor, a gastric cancer, a colon cancer, a lung cancer, a breast cancer, a germ cell tumor, an ovarian cancer, a uterine cancer, a thyroid cancer, a liver cancer, a renal cancer, a Kaposi's sarcoma, a sarcoma, a hepatocellular carcinoma, a Wilms' tumor, an acute myelogenous leukemia (AML), a myelodysplastic syndrome (MDS), an a non-small cell lung cancer (NSCLC), a myeloma, pancreatic cancer, colorectal cancer, a mesothelioma, glioblastoma, a soft tissue sarcoma, a neuroblastoma, and a nephroblastoma. The pharmaceutical composition comprising the TMMP homodimer also may be administered to such patients in a neoadjuvant or adjuvant protocol, either with or without co-administration of a checkpoint inhibitor.

The present disclosure provides a method of treating cancer in an individual, the method comprising administering to the individual an effective amount of a TMMP, where the TMMP comprises a T-cell epitope that is a WT-1 cancer-associated epitope. In some cases, an “effective amount” of a TMMP is an amount that, when administered in one or more doses to an individual in need thereof. reduces the number of cancer cells or the size of the tumor in the individual. In some cases, an “effective amount” of a TMMP is an amount that, when administered in one or more doses to an individual in need thereof, increases progression-free survival time or survival of the individual, compared to the expected progression-free survival time or survival of the individual in the absence of administration with the TMMP.

In some cases, an “effective amount” of a TMMP is an amount that, when administered in one or more doses to an individual in need thereof, either as a monotherapy or as part of a combination therapy (e.g., as part of a combination therapy with an immune checkpoint inhibitor), as discussed below, reduces the overall tumor burden in the individual, i.e., the amount of cancer in the body, or alternatively, causes the total tumor burden in the patient to remain relatively stable for a sufficient period of time for the patient to have a confirmed “stable disease” as determined by standard RECIST criteria. See, e.g., Aykan and Özatli (2020) World J. Clin. Oncol. 11:53.

In some cases, an effective amount of a TMMP is an amount that, when administered in one or more doses to an individual in need thereof, either as a monotherapy or as part of a combination therapy, e.g., with an immune checkpoint inhibitor, as discussed below, causes the tumor size to be reduced by a sufficient amount, and for a sufficient period of time, for the patient to have a confirmed “partial response” as determined by standard RECIST criteria.

In some cases, an effective amount of a TMMP is an amount that, when administered in one or more doses to an individual in need thereof, either as a monotherapy or as part of a combination therapy, e.g., with an immune checkpoint inhibitor, causes the tumor size to be reduced by a sufficient amount, and for a sufficient period of time, for the patient to have a confirmed “complete response” as determined by standard RECIST criteria.

Subjects suitable for treatment with TMMPs described herein thus include individuals who have cancer, including individuals who have been diagnosed as having cancer, individuals who have been treated for cancer but who failed to respond to the treatment, and individuals who have been treated for cancer and who initially responded but subsequently became refractory to the treatment. The following are exemplary treatments for a homodimer comprising two copies of the 1715Δ+2380 heterodimer, linked by 2 disulfide bonds between the IgG1 Fc polypeptide present in the 1715Δ polypeptides.

### Colorectal Cancer

A pharmaceutical composition comprising the TMMP homodimer can be administered to patients who have colorectal cancer. A pharmaceutical composition comprising the TMMP can be administered to patients who have colorectal cancer that has progressed following at least 2 prior systemic therapies, i.e., the TMMP will be 3rd line therapy or greater. The prior approved therapies can include one, two, three or all four of the following treatments: a fluoropyrimidine, oxaliplatin, irinotecan, bevacizumab, and, if the cancer is associated with a KRAS mutation, the prior treatment(s) can include cetuximab and/or panitumumab. The TMMP also may be administered following prior use of and failure after regorafenib and/or trifluridine/tipiracil. The pharmaceutical composition comprising the TMMP homodimer may be co-administered with a checkpoint inhibitor such as those described above. The pharmaceutical composition comprising the TMMP homodimer also may be administered to such patients in a neoadjuvant or adjuvant protocol, either with or without co-administration of a checkpoint inhibitor.

Accordingly, in an embodiment, a pharmaceutical composition comprising the TMMP homodimer administered to patients who have colorectal cancer that has progressed following treatment with at least one, at least two, at least three or all four therapeutic agents selected from a fluoropyrimidine, oxaliplatin, irinotecan and bevacizumab, and, if the cancer is associated with a KRAS mutation, cetuximab and/or panitumumab. In an embodiment, a pharmaceutical composition comprising the TMMP homodimer administered to patients who have colorectal cancer that has progressed following treatment with regorafenib and/or trifluridine/tipiracil. In an embodiment, a pharmaceutical composition comprising the TMMP homodimer administered to patients who have colorectal cancer that has progressed following treatment with at least one, at least two, at least three or all four therapeutic agents selected from a fluoropyrimidine, oxaliplatin, irinotecan and bevacizumab. In an embodiment, a pharmaceutical composition comprising the TMMP homodimer administered to patients who have colorectal cancer that is associated with a KRAS mutation and has progressed following treatment with one or both of cetuximab and panitumumab. In an embodiment, a pharmaceutical composition comprising the TMMP homodimer administered to patients who have colorectal cancer that has progressed following treatment with regorafenib and/or trifluridine/tipiracil. In an embodiment, a pharmaceutical composition comprising the TMMP homodimer administered to patients who have colorectal cancer that has progressed following treatment with at least one, at least two, at least three or all four therapeutic agents selected from a fluoropyrimidine, oxaliplatin, irinotecan and bevacizumab, and whose cancer also has progressed following treatment with regorafenib and/or trifluridine/tipiracil. In an embodiment, a pharmaceutical composition comprising the TMMP homodimer administered to patients who have colorectal cancer that has progressed following treatment with one, more than one, or all of the following: a fluoropyrimidine, oxaliplatin, irinotecan, bevacizumab, and has progressed following treatment with one or both of cetuximab and panitumumab, and has progressed following treatment with regorafenib and/or trifluridine/tipiracil. Alternatively, a pharmaceutical composition comprising the TMMP homodimer is administered to patients who have colorectal cancer, wherein the TMMP pharmaceutical composition is co-administered with one or more other therapeutic agents selected from a fluoropyrimidine, oxaliplatin, irinotecan, bevacizumab, cetuximab, panitumumab, regorafenib and/or trifluridine/tipiracil.

In each of the foregoing colorectal cancer embodiments, the amount of TMMP homodimer administered is selected from 0.5 to 1 mg/kg body weight, 1 mg/kg body weight to 5 mg/kg, and from 5 mg/kg body weight to 10 mg/kg body weight, e.g., 1 mg/kg body weight, 2 mg/kg body weight, 3 mg/kg body weight, 4 mg/kg bodyweight, 5 mg/kg body weight, 6 mg/kg body weight or 8 mg/kg body weight. In some cases, the amount of TMMP homodimer administered is 2 mg/kg. In some cases, the amount of TMMP homodimer administered is 4 mg/kg. In some instances, the amount of TMMP homodimer administered is 3 mg/kg. In each of the foregoing embodiments, the pharmaceutical composition is administered on a schedule of once a week, once every two weeks, once every three weeks, once every four weeks, once per month, or less frequently than once per month. For example, the pharmaceutical composition may be administered once every three weeks. In each of the foregoing embodiments, the pharmaceutical composition may be co-administered with at least one therapeutic agent, including, e.g., a checkpoint inhibitor such as an anti-PD-1 antibody or polypeptide, an anti-CTLA-4 antibody or polypeptide, an anti-PD-L1 antibody or polypeptide, and/or an anti-TIGIT antibody or polypeptide. For example, where the dosing schedule for pembrolizumab or other checkpoint inhibitor (e.g., an anti-PD-1) is once every three weeks, the pharmaceutical composition comprising the TMMP may be administered on the same day. For example, 2 mg/kg, 3 mg/kg or 4 mg/kg of the TMMP homodimer can be administered once every three weeks on the same day as the checkpoint inhibitor, e.g., an anti-PD1 antibody such as pembrolizumab (Keytruda®), nivolumab (Opdivo®), or cemiplimab (Libtayo®). The pharmaceutical composition comprising the TMMP homodimer also can be administered to patients in a neoadjuvant or adjuvant protocol, either with or without co-administration of a checkpoint inhibitor.

### Gastric Cancer

A pharmaceutical composition comprising the TMMP homodimer can be administered to patients who have gastric cancer. A pharmaceutical composition comprising the TMMP homodimer can be administered to patients who have gastric cancer that has progressed following at least 1 prior systemic therapy, i.e., the therapy with the TMMP will be 2nd line therapy or greater. A pharmaceutical composition comprising the TMMP homodimer can be administered to patients who have gastric cancer, including patients with high levels of microsatellite instability (MSI-H) or high tumor mutational burden, that has progressed following prior treatment with immune checkpoint inhibitor. A pharmaceutical composition comprising the TMMP homodimer can be administered to patients who have HER2+gastric cancer that have progressed following prior treatment with an anti-HER2 antibody-topoisomerase inhibitor conjugate (e.g., Fam-trastuzumab deruxtecan-nxki) in the relapse setting. A pharmaceutical composition comprising the TMMP homodimer can be administered to patients who have gastric cancer that harbors neurotrophic tyrosine receptor kinase (NTRK) gene fusions and that have progressed following prior treatment an Entrectinib class tyrosine kinase inhibitor (TKI). For each of the following therapies, the pharmaceutical composition comprising the TMMP homodimer may be co-administered with a checkpoint inhibitor such as those described above. The pharmaceutical composition comprising the TMMP homodimer also may be administered to such patients in a neoadjuvant or adjuvant protocol, either with or without co-administration of a checkpoint inhibitor.

Accordingly, in an embodiment, the TMMP homodimer administered to patients who have gastric cancer, including patients with MSI-H or high tumor mutational burden, that have progressed following treatment with one or more immune checkpoint inhibitors. In an embodiment, the TMMP homodimer administered to patients who have gastric cancer that are HER2+ and whose cancer has progressed after treatment with a HER2-directed antibody and topoisomerase inhibitor conjugated (e.g., fam-trastuzumab deruxtecan-nxki, tradename Enhertu®) in the relapse setting. In an embodiment, the TMMP homodimer administered to patients who have gastric cancer and whose cancers harbor NTRK gene fusions and whose cancers have progressed following treatment with an Entrectinib class TKI. In an embodiment, the TMMP homodimer administered to patients who have gastric cancer and whose cancer has progressed following treatment with one, two or all three therapeutic agents selected from an immune checkpoint inhibitor, a HER2-directed antibody and topoisomerase inhibitor conjugated, and an Entrectinib class TKI. Alternatively, a pharmaceutical composition comprising the TMMP homodimer is administered to patients who have gastric cancer, wherein the TMMP pharmaceutical composition is co-administered with one or more other therapeutic agents selected from an immune checkpoint inhibitor, a HER2-directed antibody and topoisomerase inhibitor conjugated, and an Entrectinib class TKI.

In each of the foregoing gastric cancer embodiments, the amount of TMMP homodimer administered is selected from 0.5 to 1 mg/kg body weight, 1 mg/kg body weight to 5 mg/kg, and from 5 mg/kg body weight to 10 mg/kg body weight, e.g., 1 mg/kg body weight, 2 mg/kg body weight, 3 mg/kg body weight, 4 mg/kg bodyweight, 5 mg/kg body weight, 6 mg/kg body weight, 7 mg/kg body weight or 8 mg/kg body weight. In each of the foregoing embodiments, the pharmaceutical composition is administered on a schedule selected from once a week, once every two weeks, once every three weeks, once every four weeks, once per month, or less frequently than once per month. For example, 2 mg/kg, 3 mg/kg or 4 mg/kg of the TMMP homodimer may be administered once every three weeks. In each of the foregoing embodiments, the pharmaceutical composition may be co-administered with at least one therapeutic agent, including, e.g., a checkpoint inhibitor such as an anti-PD-1 antibody or polypeptide, an anti-CTLA-4 antibody or polypeptide, an anti-PD-L1 antibody or polypeptide, and/or an anti-TIGIT antibody or polypeptide. For example, where the dosing schedule for pembrolizumab or other checkpoint inhibitor (e.g., an anti-PD-1) is once every three weeks, the pharmaceutical composition comprising the TMMP may be administered on the same day. For example, 2 mg/kg, 3 mg/kg or 4 mg/kg of the TMMP homodimer can be administered once every three weeks on the same day as the checkpoint inhibitor, e.g., an anti-PD1 antibody such as pembrolizumab (Keytruda®), nivolumab (Opdivo®) or cemiplimab (Libtayo®). The pharmaceutical composition comprising the TMMP homodimer also can be administered to patients in a neoadjuvant or adjuvant protocol, either with or without co-administration of a checkpoint inhibitor.

### Pancreatic Cancer

The TMMP can be administered to patients who have pancreatic cancer that has progressed following at least 1 prior systemic therapy, i.e., the TMMP will be 2^{nd} line therapy or greater. Prior systemic treatment must include either a fluoropyrimidine-based or gemcitabine-based regimen in either the adjuvant or relapsed setting. For each of the following therapies, the pharmaceutical composition comprising the TMMP homodimer may be co-administered with a checkpoint inhibitor such as those described above. The pharmaceutical composition comprising the TMMP homodimer also may be administered to such patients in a neoadjuvant or adjuvant protocol, either with or without co-administration of a checkpoint inhibitor.

Accordingly, in an embodiment, the TMMP homodimer administered to patients who have pancreatic cancer, and whose cancer has progressed following prior treatment selected from a fluoropyrimidine-based regime, a gemcitabine-based regimen, or both, in either the adjuvant or relapsed setting. Alternatively, a pharmaceutical composition comprising the TMMP homodimer is administered to patients who have pancreatic cancer, wherein the TMMP pharmaceutical composition is co-administered with one or more other therapeutic agents selected from a fluoropyrimidine-based regime and gemcitabine-based regimen, in either the adjuvant or relapsed setting.

In each of the foregoing pancreatic cancer embodiments, the amount of TMMP homodimer administered is selected from 0.5 to 1 mg/kg body weight, 1 mg/kg body weight to 5 mg/kg, and from 5 mg/kg body weight to 10 mg/kg body weight, e.g., 1 mg/kg body weight, 2 mg/kg body weight, 3 mg/kg body weight, 4 mg/kg bodyweight, 5 mg/kg body weight, 6 mg/kg body weight, 7 mg/kg body weight, or 8 mg/kg body weight. In each of the foregoing embodiments, the pharmaceutical composition is administered on a schedule selected from once a week, once every two weeks, once every three weeks, once every four weeks, once per month, or less frequently than once per month. For example, the pharmaceutical composition may be administered once every three weeks. In each of the foregoing embodiments, the pharmaceutical composition may be co-administered with at least one therapeutic agent, including, e.g., a checkpoint inhibitory therapeutic such as an anti-PD-1 antibody or polypeptide, an anti-CTLA-4 antibody or polypeptide, an anti-PD-L1 antibody or polypeptide, and/or an anti-TIGIT antibody or polypeptide. For example, where the dosing schedule for pembrolizumab or other checkpoint inhibitor (e.g., an anti-PD-1) is once every three weeks, the pharmaceutical composition comprising the TMMP may be administered on the same day. For example, 2 mg/kg, 3 mg/kg or 4 mg/kg of the TMMP homodimer can be administered once every three weeks on the same day as the checkpoint inhibitor, e.g., an anti-PD1 antibody such as pembrolizumab (Keytruda®), nivolumab (Opdivo®), or cemiplimab (Libtayo®). The pharmaceutical composition comprising the TMMP homodimer also can be administered to patients in a neoadjuvant or adjuvant protocol, either with or without co-administration of a checkpoint inhibitor.

### Ovarian Cancer

The TMMP can be administered to patients who have ovarian cancer. The TMMP can be administered to patients who have ovarian cancer that has progressed following at least 1 prior systemic therapy, i.e., in some cases, the TMMP will be 2^{nd} line therapy or greater. Prior treatment can include, e.g., one or more of in either the adjuvant or relapsed setting. Chemotherapeutic agents for treatment of ovarian cancer include, e.g., Alkeran (Melphalan), carboplatin, cisplatin, cyclophosphamide, doxorubicin hydrochloride, doxil (doxorubicin hydrochloride liposome), gemcitabine hydrochloride, hycamtin (topotecan hydrochloride), lynparza (Olaparib), melphalan, niraparib tosylate monohydrate, paclitaxel, rubraca (rucapaib camsylate), and tepadina. Therapeutic antibodies for treatment of ovarian cancer include Zirabev (Bevacizumab). In some cases, the prior treatment is cisplatin. In some cases, the prior treatment is carboplatin. In some cases, the prior treatment is a combination of carboplatin and paclitaxel. In some cases, the prior treatment is a combination of platinum and paclitaxel. For each of the following therapies, the pharmaceutical composition comprising the TMMP homodimer may be co-administered with a checkpoint inhibitor such as those described above. The pharmaceutical composition comprising the TMMP homodimer also may be administered to such patients in a neoadjuvant or adjuvant protocol, either with or without co-administration of a checkpoint inhibitor.

Accordingly, in an embodiment, the TMMP homodimer administered to patients who have ovarian cancer, and whose cancer has progressed following prior treatment with one or more of (i) surgery, (ii) chemotherapy, and (iii) antibody therapy, in either the adjuvant or relapsed setting. Disease progression can be determined using RECIST criteria. In an embodiment, the TMMP homodimer administered to patients who have ovarian cancer, and whose cancer has progressed following prior treatment with one or more cancer chemotherapeutic agents selected from Alkeran (Melphalan), carboplatin, cisplatin, cyclophosphamide, doxorubicin hydrochloride, doxil (doxorubicin hydrochloride liposome), gemcitabine hydrochloride, hycamtin (topotecan hydrochloride), lynparza (Olaparib), melphalan, niraparib tosylate monohydrate, paclitaxel, rubraca (rucapaib camsylate), and tepadina. Therapeutic antibodies for treatment of ovarian cancer include Zirabev (Bevacizumab).

In each of the foregoing ovarian cancer treatment embodiments, the amount of TMMP homodimer administered is selected from 0.5 to 1 mg/kg body weight, 1 mg/kg body weight to 5 mg/kg, and from 5 mg/kg body weight to 10 mg/kg body weight, e.g., 1 mg/kg body weight, 2 mg/kg body weight, 3 mg/kg body weight, 4 mg/kg bodyweight, 5 mg/kg body weight, 6 mg/kg body weight, 7 mg/kg body weight, or 8 mg/kg body weight. In each of the foregoing embodiments, the pharmaceutical composition is administered on a schedule selected from once a week, once every two weeks, once every three weeks, once every four weeks, once per month, or less frequently than once per month. For example, the pharmaceutical composition may be administered once every three weeks. In each of the foregoing embodiments, the pharmaceutical composition may be co-administered with at least one therapeutic agent, including, e.g., a checkpoint inhibitory therapeutic such as an anti-PD-1 antibody or polypeptide, an anti-CTLA-4 antibody or polypeptide, an anti-PD-L1 antibody or polypeptide, and/or an anti-TIGIT antibody or polypeptide. For example, where the dosing schedule for pembrolizumab or other checkpoint inhibitor (e.g., an anti-PD-1) is once every three weeks, the pharmaceutical composition comprising the TMMP may be administered on the same day. For example, 2 mg/kg, 3 mg/kg or 4 mg/kg of the TMMP homodimer can be administered once every three weeks on the same day as the checkpoint inhibitor, e.g., an anti-PD1 antibody such as pembrolizumab (Keytruda®), nivolumab (Opdivo®), or cemiplimab (Libtayo®). The pharmaceutical composition comprising the TMMP homodimer also can be administered to patients in a neoadjuvant or adjuvant protocol, either with or without co-administration of a checkpoint inhibitor.

### Glioblastoma

The TMMP can be administered to patients who have glioblastoma (also called glioblastoma multiforme or “GBM”). The TMMP can be administered to patients who have GBM that has progressed following at least 1 prior therapy, i.e., in some cases, the TMMP will be 2^{nd} line therapy or greater. Prior treatment can include one or more of surgery, radiation treatment, and chemotherapy (e.g., with temozolomide), e.g., treatment with radiation and temozolomide.

For each of the following therapies, the pharmaceutical composition comprising the TMMP homodimer may be co-administered with a checkpoint inhibitor such as those described above. Preliminary antitumor activity will be evaluated by RANO and immune RANO (IRANO). RANO is the acronym for Response Assessment in Neuro-Oncology criteria, which is used to assess response to treatment of GMB. The pharmaceutical composition comprising the TMMP homodimer also may be administered to such patients in a neoadjuvant or adjuvant protocol, either with or without co-administration of a checkpoint inhibitor.

Accordingly, in an embodiment, the TMMP homodimer administered to patients who have GBM, and whose cancer has progressed following prior treatment selected from a surgery, radiation treatment, chemotherapy, or all three, in either the adjuvant or relapsed setting. In an embodiment, the TMMP homodimer administered to patients who have GBM, and whose cancer has progressed following prior treatment with radiation and temozolomide (including one or more therapies following treatment with radiation and temozolomide).

In each of the foregoing GMB treatment embodiments, the amount of TMMP homodimer administered is selected from 0.5 to 1 mg/kg body weight, 1 mg/kg body weight to 5 mg/kg, and from 5 mg/kg body weight to 10 mg/kg body weight, e.g., 1 mg/kg body weight, 2 mg/kg body weight, 3 mg/kg body weight, 4 mg/kg bodyweight, 5 mg/kg body weight, 6 mg/kg body weight, 7 mg/kg body weight, or 8 mg/kg body weight. In each of the foregoing embodiments, the pharmaceutical composition is administered on a schedule selected from once a week, once every two weeks, once every three weeks, once every four weeks, once per month, or less frequently than once per month. For example, the pharmaceutical composition may be administered once every three weeks. In each of the foregoing embodiments, the pharmaceutical composition may be co-administered with at least one therapeutic agent, including, e.g., a checkpoint inhibitory therapeutic such as an anti-PD-1 antibody or polypeptide. an anti-CTLA-4 antibody or polypeptide, an anti-PD-L1 antibody or polypeptide, and/or an anti-TIGIT antibody or polypeptide. For example, where the dosing schedule for pembrolizumab or other checkpoint inhibitor (e.g., an anti-PD-1) is once every three weeks, the pharmaceutical composition comprising the TMMP may be administered on the same day. For example, 2 mg/kg, 3 mg/kg or 4 mg/kg of the TMMP homodimer can be administered once every three weeks on the same day as the checkpoint inhibitor, e.g., an anti-PD1 antibody such as pembrolizumab (Keytruda®), nivolumab (Opdivo®), or cemiplimab (Libtayo®). The pharmaceutical composition comprising the TMMP homodimer also can be administered to patients in a neoadjuvant or adjuvant protocol, either with or without co-administration of a checkpoint inhibitor.

### Acute Myeloid Leukemia

The TMMP can be administered to patients who have acute myeloid leukemia (AML). The TMMP can be administered to patients who have AML that has progressed following at least 1 prior therapy (e.g., after at least 2 cycles of frontline chemotherapy), i.e., in some cases, the TMMP will be 2^{nd} line therapy or greater. Prior treatment can include, e.g., one or more of (i) a combination of cytarabine (cytosine arabinoside) and an anthracycline drug (e.g., daunorubicin or idarubicin); (ii) a FLT3 inhibitor such as midostaurin (Rydapt) or gilteritinib (Xospata); (iii) an isocitrate dehydrogenase (IDH) inhibitor such as ivosidenib (Tibsovo) or enasidenib (Idhifa); (iv) gemtuzumab ozogamicin (Mylotarg); (v) a BCL-2 inhibitor such as ventoclax (Venclext); or (v) a hedgehog pathway inhibitor such as glasdegib (Daurismo) in either the adjuvant or relapsed setting. Patients who underwent prior allogeneic hematopoietic stem cell are not precluded provided they have recovered from acute toxicités of transplant, are greater than one hundred days from stem cell infusion and have no active grade 3 or grade 4 graft versus host disease (GVHD). For each of the following therapies, the pharmaceutical composition comprising the TMMP homodimer may be co-administered with a checkpoint inhibitor such as those described above. The pharmaceutical composition comprising the TMMP homodimer also may be administered to such patients in a neoadjuvant or adjuvant protocol, either with or without co-administration of a checkpoint inhibitor.

Accordingly, in an embodiment, the TMMP homodimer administered to patients who have AML, and whose cancer has progressed following prior treatment with one or more of (i) a combination of cytarabine (cytosine arabinoside) and an anthracycline drug (e.g., daunorubicin or idarubicin); (ii) a FLT3 inhibitor such as midostaurin (Rydapt) or gilteritinib (Xospata); (iii) an IDH inhibitor such as ivosidenib (Tibsovo) or enasidenib (Idhifa); (iv) gemtuzumab ozogamicin (Mylotarg); (v) a BCL-2 inhibitor such as ventoclax (Venclext); or (v) a hedgehog pathway inhibitor such as glasdegib (Daurismo) in either the adjuvant or relapsed setting. Disease progression can be determined using ELN (European Leukemia Network) criteria. In an embodiment, the TMMP homodimer administered to patients who have AML, and whose cancer has progressed following prior treatment with cytosine arabinoside and daunorubicin (including one or more therapies following treatment with cytosine arabinoside and daunorubicin).

In each of the foregoing AML treatment embodiments, the amount of TMMP homodimer administered is selected from 0.5 to 1 mg/kg body weight, 1 mg/kg body weight to 5 mg/kg, and from 5 mg/kg body weight to 10 mg/kg body weight, e.g., 1 mg/kg body weight, 2 mg/kg body weight, 3 mg/kg body weight, 4 mg/kg bodyweight, 5 mg/kg body weight, 6 mg/kg body weight, 7 mg/kg body weight, or 8 mg/kg body weight. In each of the foregoing embodiments, the pharmaceutical composition is administered on a schedule selected from once a week, once every two weeks, once every three weeks, once every four weeks, once per month, or less frequently than once per month. For example, the pharmaceutical composition may be administered once every three weeks. In each of the foregoing embodiments, the pharmaceutical composition may be co-administered with at least one therapeutic agent, including, e.g., a checkpoint inhibitory therapeutic such as an anti-PD-1 antibody or polypeptide. an anti-CTLA-4 antibody or polypeptide, an anti-PD-L1 antibody or polypeptide, and/or an anti-TIGIT antibody or polypeptide. For example, where the dosing schedule for pembrolizumab or other checkpoint inhibitor (e.g., an anti-PD-1) is once every three weeks, the pharmaceutical composition comprising the TMMP may be administered on the same day. For example, 2 mg/kg, 3 mg/kg or 4 mg/kg of the TMMP homodimer can be administered once every three weeks on the same day as the checkpoint inhibitor, e.g., an anti-PD1 antibody such as pembrolizumab (Keytruda®), nivolumab (Opdivo®), or cemiplimab (Libtayo®). The pharmaceutical composition comprising the TMMP homodimer also can be administered to patients in a neoadjuvant or adjuvant protocol, either with or without co-administration of a checkpoint inhibitor.

### Triple-Negative Breast Cancer

The TMMP can be administered to patients who have triple-negative breast cancer; i.e., breast cancer that is estrogen receptor-negative, progesterone receptor-negative, and HER2-negative. The TMMP can be administered to patients who have triple-negative breast cancer that has progressed following at least 1 prior systemic therapy, i.e., the TMMP will be 2^{nd} line therapy or greater. Prior treatment can include: (i) surgery; (ii) olaparib (Lynparza); and (iii) capecitabine (Xeloda), alone or in combination with pembrolizumab); in either the adjuvant or relapsed setting. For each of the following therapies, the pharmaceutical composition comprising the TMMP homodimer may be co-administered with a checkpoint inhibitor such as those described above. The pharmaceutical composition comprising the TMMP homodimer also may be administered to such patients in a neoadjuvant or adjuvant protocol, either with or without co-administration of a checkpoint inhibitor.

Accordingly, in an embodiment, the TMMP homodimer administered to patients who have triple-negative breast cancer, and whose cancer has progressed following one or more prior therapies selected from (i) surgery; (ii) olaparib (Lynparza); and/or (iii) capecitabine (Xeloda), each of te foregoing alone or in combination with pembrolizumab; in either the adjuvant or relapsed setting, including one or more therapies following one or more of surgery, olaparib (Lynparza, and/or capecitabine (Xeloda).

In each of the foregoing triple-negative breast cancer treatment embodiments, the amount of TMMP homodimer administered is selected from 0.5 to 1 mg/kg body weight, 1 mg/kg body weight to 5 mg/kg, and from 5 mg/kg body weight to 10 mg/kg body weight, e.g., 1 mg/kg body weight, 2 mg/kg body weight, 3 mg/kg body weight, 4 mg/kg bodyweight, 5 mg/kg body weight, 6 mg/kg body weight, 7 mg/kg body weight, or 8 mg/kg body weight. In each of the foregoing embodiments, the pharmaceutical composition is administered on a schedule selected from once a week, once every two weeks, once every three weeks, once every four weeks, once per month, or less frequently than once per month. For example, the pharmaceutical composition may be administered once every three weeks. In each of the foregoing embodiments, the pharmaceutical composition may be co-administered with at least one therapeutic agent, including, e.g., a checkpoint inhibitory therapeutic such as an anti-PD-1 antibody or polypeptide, an anti-CTLA-4 antibody or polypeptide, an anti-PD-L1 antibody or polypeptide, and/or an anti-TIGIT antibody or polypeptide. For example, where the dosing schedule for pembrolizumab or other checkpoint inhibitor (e.g., an anti-PD-1) is once every three weeks, the pharmaceutical composition comprising the TMMP may be administered on the same day. For example, 2 mg/kg, 3 mg/kg or 4 mg/kg of the TMMP homodimer can be administered once every three weeks on the same day as the checkpoint inhibitor, e.g., an anti-PD1 antibody such as pembrolizumab (Keytruda®), nivolumab (Opdivo®), or cemiplimab (Libtayo®). The pharmaceutical composition comprising the TMMP homodimer also can be administered to patients in a neoadjuvant or adjuvant protocol, either with or without co-administration of a checkpoint inhibitor.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-38 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

| No. | Aspect |
|---|---|
| 1. | A method of treating a patient having colorectal cancer, gastric cancer or pancreatic cancer |
| | that has progressed following treatment with a prior therapeutic agent, wherein the method |
| | comprises administering to the patient an effective amount of a pharmaceutical composition |
| | comprising a TMMP, wherein the TMMP comprises a homodimer comprising two |
| | heterodimeric TMMPs, each TMMP comprising: a) a first polypeptide comprising: i) a WT-1 |
| | peptide epitope; and ii) a beta-2 microglobulin (β2M); b) a second polypeptide comprising i) a |
| | class I MHC heavy chain polypeptide; ii) at least one variant IL-2 MOD, and iii) an |
| | immunoglobulin (Ig) Fc polypeptide. |
| 2. | A method according to Aspect 1, wherein the patient has colorectal cancer that has progressed |
| | following treatment with one or more, or all of the following: a fluoropyrimidine, oxaliplatin, |
| | irinotecan, bevacizumab |
| 3. | A method according to Aspect 1 or 2, wherein the patient has colorectal cancer associated |
| | with a KRAS mutation, and the cancer has progressed following treatment with one or more, |
| | or cetuximab or panitumumab. |
| 4. | A method according to any one of Aspects 1-3, wherein the patient has colorectal cancer that |
| | has progressed following treatment with regorafenib and/or trifluridine/tipiracil. |
| 5. | A method according to Aspect 1, wherein the patient has colorectal cancer that has progressed |
| | following treatment with one, more than one, or all of the following: a fluoropyrimidine, |
| | oxaliplatin, irinotecan, bevacizumab, and has progressed following treatment with one or both |
| | of cetuximab and panitumumab, and has progressed following treatment with regorafenib |
| | and/or trifluridine/tipiracil. |
| 6. | A method according to Aspect 1, wherein the patient has gastric cancer and has MSI-H or |
| | high tumor mutational burden, and wherein the cancer has progressed following treatment |
| | with one or more immune checkpoint inhibitors. |
| 7. | A method according to Aspect 1 or Aspect 6, wherein the patient has gastric cancer that is |
| | HER2+ and the cancer has progressed in the relapse setting after treatment with a |
| | HER2-directed antibody and topoisomerase inhibitor conjugated. |
| 8. | A method according to any one of Aspects 1, 6 or 7, wherein the patient has gastric cancer that |
| | harbors a NTRK gene fusion and the cancer has progressed following treatment with an |
| | Entrectinib class TKI. |
| 9. | A method according to Aspect 1, wherein the patient has gastric cancer that has progressed |
| | following treatment with each of an immune checkpoint inhibitor, a HER2-directed antibody |
| | and topoisomerase inhibitor conjugated, and an Entrectinib class TKI. |
| 10. | A method according to Aspect 1, wherein the patient has pancreatic cancer, and wherein the |
| | cancer has progressed following prior treatment selected from a fluoropyrimidine-based |
| | regime, a gemcitabine-based regimen, or both, in either the adjuvant or relapsed setting. |
| 11. | A method of treating a patient having a cancer according to any one of Aspects 1-10, wherein |
| | the amount of TMMP homodimer administered is selected from 0.5 to 1 mg/kg body weight, 1 |
| | mg/kg body weight to 5 mg/kg, and from 5 mg/kg body weight to 10 mg/kg body weight. |
| 12. | A method of treating a patient having a cancer according to any one of Aspects 1-10, wherein |
| | the amount of TMMP homodimer administered is 1 mg/kg body weight. |
| 13. | A method of treating a patient having a cancer according to any one of Aspects 1-10, wherein |
| | the amount of TMMP homodimer administered is 2 mg/kg body weight. |
| 14. | A method of treating a patient having a cancer according to any one of Aspects 1-10, wherein |
| | the amount of TMMP homodimer administered is 3 mg/kg body weight. |
| 15. | A method of treating a patient having a cancer according to any one of Aspects 1-10, wherein |
| | the amount of TMMP homodimer administered is 4 mg/kg bodyweight. |
| 16. | A method of treating a patient having a cancer according to any one of Aspects 1-10, wherein |
| | the amount of TMMP homodimer administered is 5 mg/kg body weight. |
| 17. | A method of treating a patient having a cancer according to any one of Aspects 1-10, wherein |
| | the amount of TMMP homodimer administered is 6 mg/kg body weight. |
| 18. | A method of treating a patient having a cancer according to any one of Aspects 1-10, wherein |
| | the amount of TMMP homodimer administered is 7 mg/kg body weight. |
| 19. | A method of treating a patient having a cancer according to any one of Aspects 1-10, wherein |
| | the amount of TMMP homodimer administered is 8 mg/kg body weight. |
| 20. | A method of treating a patient having a cancer according to any one of Aspects 11-19, |
| | wherein the pharmaceutical composition is administered once a week, once every two weeks, |
| | once every three weeks, once every four weeks, or once per month, or less frequently than |
| | once per month, and wherein administration is continued until disease progression or |
| | unacceptable toxicity. |
| 21. | A method of treating a patient having a cancer according to any one of Aspects 11-19, |
| | wherein the pharmaceutical composition is administered once every three weeks, and wherein |
| | administration is continued until disease progression or unacceptable toxicity. |
| 22. | A method of treating a patient having a cancer according to any one of Aspects 11-21, |
| | wherein the pharmaceutical composition is co-administered with at least one therapeutic |
| | agent. |
| 23. | A method of treating a patient having a cancer according to any one of Aspects 11-22, |
| | wherein the pharmaceutical composition is co-administered with at least one immune |
| | checkpoint inhibitors. |
| 24. | A method according to Aspect 23, wherein the at least one immune checkpoint inhibitor is an |
| | antibody that binds to a polypeptide selected from CD27, CD28, CD40, CD122, CD96, CD73, |
| | CD47, OX40, GITR, CSF1R, JAK, PI3K delta, PI3K gamma, TAM, arginase, CD137, ICOS, |
| | A2AR, B7-H3, B7-H4, BTLA, CTLA-4, LAG3, TIM3, VISTA, CD96, TIGIT, CD122, PD-1, |
| | PD-L1, and PD-L2. |
| 25. | A method according to Aspect 23, wherein the at least one immune checkpoint inhibitor is |
| | selected from nivolumab, pembrolizumab, cemiplimab, pidilizumab, AMP-224, |
| | MPDL3280A, MDX-1105, MEDI-4736, arelumab, ipilimumab, tremelimumab, pidilizumab, |
| | IMP321, MGA271, BMS-986016, lirilumab, urelumab, PF-05082566, IPH2101, MEDI-6469, |
| | CP-870, 893, Mogamulizumab, Varlilumab, Avelumab, Galiximab, AMP-514, AUNP 12, |
| | Indoximod, NLG-919, INCB024360, KN035, and combinations thereof. |
| 26. | A method according to Aspect 23, wherein the at least one immune checkpoint inhibitor is |
| | selected from an anti-PD-1 antibody or polypeptide, an anti-CTLA-4 antibody or polypeptide, |
| | an anti-PD-L1 antibody or polypeptide, and an anti-TIGIT antibody or polypeptide. |
| 27. | A method according to Aspect 23, wherein the at least one immune checkpoint inhibitor is an |
| | anti-PD-1 antibody. |
| 28. | A method according to any one of Aspects 1-27, wherein each heterodimeric polypeptide |
| | comprises: a) a first polypeptide comprising: i) a WT-1 peptide epitope; and ii) a first MHC |
| | polypeptide, which is a beta-2 microglobulin (β2M); b) a second polypeptide comprising i) a |
| | second MHC polypeptide, which is a class I MHC heavy chain polypeptide; ii) two variant |
| | IL-2 MODs in tandem, and iii) an immunoglobulin (Ig) Fc polypeptide. |
| 29. | A method according to any one of Aspects 1-27, wherein each heterodimeric polypeptide |
| | comprises: |
| | a1) a first polypeptide comprising, in order from N-terminus to C-terminus: i) a WT-1 |
| | epitope, ii) a Cys-containing peptide linker, and iii) a β2M polypeptide; and |
| | b1) a second polypeptide comprising, in order from N-terminus to C-terminus: i) a first |
| | variant IL-2 polypeptide (MOD), ii) a peptide linker, iii) a second variant IL-2 polypeptide |
| | (MOD), iv) a peptide linker, v) a class I MHC heavy chain polypeptide, v) a peptide linker; |
| | and vi) an Ig Fc polypeptide, |
| | or |
| | a2) a first polypeptide comprising, in order from N-terminus to C-terminus: i) a WT-1 |
| | epitope, ii) a Cys-containing peptide linker, and iii) a β2M polypeptide; and |
| | b2) a second polypeptide comprising, in order from N-terminus to C-terminus: i) a class I |
| | MHC heavy chain polypeptide, ii) a peptide linker; iii) an Ig Fc polypeptide, iv) a peptide |
| | linker; v) a first variant IL-2 polypeptide (MOD), vi) a peptide linker, and vii) a second variant |
| | IL-2 polypeptide. |
| 30. | A method according to any one of Aspects 1-29, wherein the Ig Fc polypeptide comprises a |
| | IgG1 Fc polypeptide that has at least about 85%, at least about 90%, at least about 95%, at |
| | least about 98%, at least about 99%, or 100%, amino acid sequence identity to SEQ ID NO: 5 |
| | (226 amino acids) or SEQ ID NO: 6 (227 amino acids). |
| 31. | A method according to any one of Aspects 1-30, wherein the Ig Fc polypeptide comprises an |
| | IgG1 Fc polypeptide that has a reduced ability to effect complement-dependent cytotoxicity |
| | (CDC) and/or antibody-dependent cell cytotoxicity (ADCC), optionally wherein the that has at |
| | least about 85%, at least about 90%, at least about 95%, at least about 98%, at least about |
| | 99%, or 100%, amino acid sequence identity to SEQ ID NO: 5 (226 amino acids) or SEQ ID |
| | NO: 6 (227 amino acids), wherein the IgG1 Fc comprises both (i) a substitution of L234 (L14 |
| | of the IgG1 Fc amino acid sequence of SEQ ID NO: 5 and SEQ ID NO: 6) with an amino acid |
| | other than leucine, e.g., alanine, and (ii) a substitution of L235 (L15 of the IgG1 Fc amino acid |
| | sequence SEQ ID NO: 5 and SEQ ID NO: 6) with an amino acid other than leucine, e.g., |
| | alanine. |
| 32. | A method according to any one of Aspects 1-31, wherein the class I MHC heavy chain is a |
| | heavy chain polypeptide has: |
| | a) an amino acid sequence having least 85%, at least 90%, at least 95%, at least 98%, at |
| | least 99%, or 100%, amino acid sequence identity to the HLA-A*0101, HLA-A*0201, |
| | HLA-A*0201, HLA-A*1101, HLA-A*2301, HLA-A*2402, HLA-A*2407, HLA-A*3303, or HLA- |
| | A*3401 amino acid sequence depicted in FIG. 4; or |
| | b) an amino acid sequence having at least 85%, at least 90%, at least 95%, at least 98%, at |
| | least 99%, or 100% amino acid sequence identity to the HLA-B*0702, HLA-B*0801, |
| | HLA-B*1502, HLA-B*3802, HLA-B*4001, HLA-B*4601, or HLA-B*5301 amino acid sequence |
| | depicted in FIG. 5; |
| | c) an amino acid sequence having at least having least 85%, at least 90%, at least 95%, at |
| | least 98%, at least 99%, or 100% sequence identity to the HLA-C*0102, HLA-C*0303, |
| | HLA-C*0304, HLA-C*0401, HLA-C*0602, HLA-C*0701, HLA-C*0702, HLA-C*0801, or |
| | HLA-C*1502 depicted in FIG. 6; or |
| | d) an HLA-E sequence having at least having least 85%, at least 90%, at least 95%, at least |
| | 98%, at least 99%, or 100% amino acid sequence identity to the sequences of FIG. 7A-D. |
| 33. | A method according to any one of Aspects 1-32, wherein the at least one variant IL-2 MOD at |
| | least 90%, at least 95%, at least 98%, or at least 99% amino acid sequence identity to the |
| | wild-type IL-2 amino acid sequence (SEQ ID NO: 54). |
| 34. | A method according to Aspect 33, wherein the at least one variant IL-2 MOD exhibits |
| | decreased or substantially no binding to IL-2Rα, and also exhibits decreased binding to |
| | IL-2Rβ and/or IL-2Rγ. |
| 35. | A method according to Aspect 33, wherein the at least one variant IL-2 MOD exhibits |
| | decreased or substantially no binding to IL-2Rα, and also exhibits decreased binding to |
| | IL-2Rβ. |
| 36. | A method according to any one of Aspects 33-35, wherein the at least one variant IL-2 MOD |
| | comprises H16A and F42A substitutions or H16T and F42A substitutions. |
| 37. | A method according to any one of Aspects 1-36, wherein the TMMP comprises two variant |
| | IL-2 MODs in tandem, optionally wherein the two variant IL-2 MODs comprises H16A and |
| | F42A substitutions, and wherein the two variant IL-2 MOD are joined by a linker. |
| 38. | A method of treating a patient having a cancer according to any one of Aspects 1-37, wherein |
| | the TMMP is a homodimer comprising two copies of the 1715Δ + 2380 heterodimer, linked |
| | by 2 disulfide bonds between the Ig Fc polypeptide present in the 1715Δ polypeptides. |

### FURTHER ASPECTS

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method of treating a patient having colorectal cancer, gastric cancer, pancreatic cancer, glioblastoma, acute myeloid leukemia (AML), or triple-negative breast cancer that has progressed following treatment with a prior therapy, wherein the method comprises administering to the patient an effective amount of a pharmaceutical composition comprising a TMMP, wherein the TMMP comprises a homodimer comprising two heterodimeric TMMPs, each TMMP comprising: a) a first polypeptide comprising: i) a WT-1 peptide epitope; and ii) a beta-2 microglobulin (β2M); b) a second polypeptide comprising i) a class I MHC heavy chain polypeptide; ii) at least one variant IL-2 MOD, and iii) an immunoglobulin (Ig) Fc polypeptide.

Aspect 2. A method according to aspect 1, wherein the patient has colorectal cancer that has progressed following treatment with one or more, or all of the following: a fluoropyrimidine, oxaliplatin, irinotecan, bevacizumab.

Aspect 3. A method according to aspect 1 or aspect 2, wherein the patient has colorectal cancer associated with a KRAS mutation, and the cancer has progressed following treatment with one or both of cetuximab and panitumumab.

Aspect 4. A method according to any one of aspects 1-3, wherein the patient has colorectal cancer that has progressed following treatment with regorafenib and/or trifluridine/tipiracil.

Aspect 5. A method according to aspect 1, wherein the patient has colorectal cancer that has progressed following treatment with one, more than one, or all of the following: a fluoropyrimidine, oxaliplatin, irinotecan, bevacizumab, and has progressed following treatment with one or both of cetuximab and panitumumab, and has progressed following treatment with regorafenib and/or trifluridine/tipiracil.

Aspect 6. A method according to aspect 1, wherein the patient has gastric cancer and has high levels of microsatellite instability (MSI-H) or high tumor mutational burden, and wherein the cancer has progressed following treatment with one or more immune checkpoint inhibitors.

Aspect 7. A method according to aspect 1 or aspect 6, wherein the patient has gastric cancer that is HER2+and the cancer has progressed in the relapse setting after treatment with a HER2-directed antibody and topoisomerase inhibitor conjugated.

Aspect 8. A method according to any one of aspects 1, 6 or 7, wherein the patient has gastric cancer that harbors a NTRK gene fusion and the cancer has progressed following treatment with an Entrectinib class tyrosine kinase inhibitor (TKI).

Aspect 9. A method according to aspect 1, wherein the patient has gastric cancer that has progressed following treatment with each of an immune checkpoint inhibitor, a HER2-directed antibody and topoisomerase inhibitor conjugated, and an Entrectinib class TKI.

Aspect 10. A method according to aspect 1, wherein the patient has pancreatic cancer, and wherein the cancer has progressed following prior treatment selected from a fluoropyrimidine-based regime, a gemcitabine-based regimen, or both, in either the adjuvant or relapsed setting.

Aspect 11. A method according to aspect 1, wherein the patient has glioblastoma that has progressed following treatment with one, two, or all of the following: surgery, radiation, and temozolomide.

Aspect 12. A method according to aspect 1, wherein the patient has ovarian cancer that has progressed following treatment with one, two, or all of the following: surgery, antibody therapy, carboplatin, and paclitaxel.

Aspect 13. A method according to aspect 1, wherein the patient has acute myelogenous leukemia (AML) that has progressed following treatment with one or more, or all of the following: (i) a combination of cytarabine and an anthracycline drug; (ii) a FLT3 inhibitor; (iii) an isocitrate dehydrogenase (IDH) inhibitor; (iv) gemtuzumab ozogamicin; (v) a BCL-2 inhibitor; and (v) a hedgehog pathway inhibitor.

Aspect 14. A method according to aspect 1, wherein the patient has triple-negative breast cancer that has progressed following treatment with one or more, or all of the following: (i) surgery; (ii) olaparib; (iii) capecitabine (Xeloda); and (iv) pembrolizumab.

Aspect 15. A method of treating a patient having a cancer according to any one of aspects 1-14, wherein the amount of TMMP homodimer administered is selected from 0.5 to 1 mg/kg body weight, 1 mg/kg body weight to 5 mg/kg, and from 5 mg/kg body weight to 10 mg/kg body weight.

Aspect 16. A method of treating a patient having a cancer according to any one of aspects 1-14, wherein the amount of TMMP homodimer administered is 1 mg/kg body weight.

Aspect 17. A method of treating a patient having a cancer according to any one of aspects 1-14, wherein the amount of TMMP homodimer administered is 2 mg/kg body weight.

Aspect 18. A method of treating a patient having a cancer according to any one of aspects 1-14, wherein the amount of TMMP homodimer administered is 3 mg/kg body weight.

Aspect 19. A method of treating a patient having a cancer according to any one of aspects 1-14, wherein the amount of TMMP homodimer administered is 4 mg/kg bodyweight.

Aspect 20. A method of treating a patient having a cancer according to any one of aspects 1-14, wherein the amount of TMMP homodimer administered is 5 mg/kg body weight.

Aspect 21. A method of treating a patient having a cancer according to any one of aspects 1-14, wherein the amount of TMMP homodimer administered is 6 mg/kg body weight.

Aspect 22. A method of treating a patient having a cancer according to any one of aspects 1-14, wherein the amount of TMMP homodimer administered is 7 mg/kg body weight.

Aspect 23. A method of treating a patient having a cancer according to any one of aspects 1-14, wherein the amount of TMMP homodimer administered is 8 mg/kg body weight.

Aspect 24. A method of treating a patient having a cancer according to any one of aspects 15-23, wherein the pharmaceutical composition is administered once a week, once every two weeks, once every three weeks, once every four weeks, or once per month, or less frequently than once per month, and wherein administration is continued until disease progression or unacceptable toxicity.

Aspect 25. A method of treating a patient having a cancer according to any one of aspects 15-23, wherein the pharmaceutical composition is administered once every three weeks, and wherein administration is continued until disease progression or unacceptable toxicity.

Aspect 26. A method of treating a patient having a cancer according to any one of aspects 15-23, wherein the pharmaceutical composition is co-administered with at least one therapeutic agent.

Aspect 27. A method of treating a patient having a cancer according to any one of aspects 15-23, wherein the pharmaceutical composition is co-administered with at least one immune checkpoint inhibitor.

Aspect 28. A method according to aspect 27, wherein the at least one immune checkpoint inhibitor is an antibody that binds to a polypeptide selected from CD27, CD28, CD40, CD122, CD96, CD73, CD47, OX40, GITR, CSFIR, JAK, PI3K delta, PI3K gamma, TAM, arginase, CD137, ICOS, A2AR, B7-H3, B7-H4, BTLA, CTLA-4, LAG3, TIM3, VISTA, CD96, TIGIT, CD122, PD-1, PD-L1, and PD-L2.

Aspect 29. A method according to aspect 27, wherein the at least one immune checkpoint inhibitor is selected from nivolumab, pembrolizumab, cemiplimab, pidilizumab, AMP-224, MPDL3280A, MDX-1105, MEDI-4736, arelumab, ipilimumab, tremelimumab, pidilizumab, IMP321, MGA271, BMS-986016, lirilumab, urelumab, PF-05082566, IPH2101, MEDI-6469, CP-870,893, Mogamulizumab, Varlilumab, Avelumab, Galiximab, AMP-514, AUNP 12, Indoximod, NLG-919, INCB024360, KN035, and combinations thereof.

Aspect 30. A method according to aspect 27, wherein the at least one immune checkpoint inhibitor is selected from an anti-PD-1 antibody or polypeptide, an anti-CTLA-4 antibody or polypeptide, an anti-PD-L1 antibody or polypeptide, and an anti-TIGIT antibody or polypeptide.

Aspect 31. A method according to aspect 27, wherein the at least one immune checkpoint inhibitor is an anti-PD-1 antibody.

Aspect 32. A method according to any one of aspects 1-31 wherein each heterodimeric polypeptide comprises: a) a first polypeptide comprising: i) a WT-1 peptide epitope; and ii) a first MHC polypeptide, which is a beta-2 microglobulin (β2M); b) a second polypeptide comprising i) a second MHC polypeptide, which is a class I MHC heavy chain polypeptide; ii) two variant IL-2 MODs in tandem, and iii) an immunoglobulin (Ig) Fc polypeptide.

Aspect 33. A method according to any one of aspects 1-32, wherein each heterodimeric polypeptide comprises: a) a first polypeptide comprising, in order from N-terminus to C-terminus: i) a WT-1 epitope, ii) a Cys-containing peptide linker, and iii) a β2M polypeptide; and b) a second polypeptide comprising, in order from N-terminus to C-terminus: i) a first variant IL-2 polypeptide (MOD), ii) a peptide linker, iii) a second variant IL-2 polypeptide (MOD), iv) a peptide linker, v) a class I MHC heavy chain polypeptide, v) a peptide linker; and vi) an Ig Fc polypeptide.

Aspect 34. A method according to any one of aspects 1-32, wherein each heterodimeric polypeptide comprises: a) a first polypeptide comprising, in order from N-terminus to C-terminus: i) a WT-1 epitope; ii) a Cys-containing peptide linker; and iii) a β2M polypeptide, and b) a second polypeptide comprising, in order from N-terminus to C-terminus: i) a class I MHC heavy chain polypeptide; ii) a peptide linker; iii) an Ig Fc polypeptide; iv) a peptide linker; v) a first variant IL-2 polypeptide (MOD); vi) a peptide linker; and vii) a second variant IL-2 polypeptide.

Aspect 35. A method according to any one of aspects 1-34, wherein the Ig Fc polypeptide comprises a IgG1 Fc polypeptide that has at least about 85%, at least about 90%, at least about 95%, at least about 98%, at least about 99%, or 100%, amino acid sequence identity to SEQ ID NO: 5 (226 amino acids) or SEQ ID NO:6 (227 amino acids), and optionally, wherein the IgG1 Fc polypeptide has a reduced ability to effect complement-dependent cytotoxicity (CDC) and/or antibody-dependent cell cytotoxicity (ADCC).

Aspect 36. A method according to any one of aspects 1-35, wherein the class I MHC heavy chain is an HLA-A heavy chain, optionally wherein the class I MHC heavy chain comprises an amino acid sequence having at least 95% amino acid sequence identity to the amino acid sequence identity to the HLA-A*0101, HLA-A*0201, HLA-A*0201, HLA-A*1101, HLA-A*2301, HLA-A*2402, HLA-A*2407, HLA-A*3303, or HLA-A*3401 amino acid sequence depicted in FIG. 4.

Aspect 37. A method according to any one of aspects 1-36, wherein the at least one variant IL-2 MOD at least 90%, at least 95%, at least 98%, or at least 99% amino acid sequence identity to the wild-type IL-2 amino acid sequence (SEQ ID NO:54).

Aspect 38. A method according to aspect 37, wherein the at least one variant IL-2 MOD exhibits decreased or substantially no binding to IL-2Rα, and also exhibits decreased binding to IL-2Rβ.

Aspect 39. A method according to aspect 37, wherein the at least one variant IL-2 MOD exhibits decreased or substantially no binding to IL-2Rα, and also exhibits decreased binding to IL-2Rγ.

Aspect 40. A method according to aspect 37, wherein the at least one variant IL-2 MOD exhibits decreased or substantially no binding to IL-2Rα, and also exhibits decreased binding to both IL-2β and IL-2Rγ.

Aspect 41. A method according to aspect 37, wherein the at least one variant IL-2 MOD comprises H16A and F42A substitutions or H16T and F42A substitutions.

Aspect 42. A method according to any one of aspects 1-41, wherein the TMMP comprises two variant IL-2 MODs in tandem.

Aspect 43. A method of treating a patient having a cancer according to any one of aspects 1-42, wherein the TMMP is a homodimer comprising two copies of the 1715Δ+2380 heterodimer, wherein each heterodimer comprises: a) a first disulfide linkage is between i) the Cys present in the linker between the WT1 peptide and the β2M chain in the 2380 polypeptide, and ii) the Cys at Y84C in the class I heavy chain present in the 1715Δ polypeptide; and b) a second disulfide linkages is between: i) the Cys at R12C in the β2M polypeptide present in the 2380 polypeptide, and ii) the Cys at A236C in the class I heavy chain present in the 1715A polypeptide, and wherein the two heterodimers are linked by two disulfide bonds between the IgG1 Fc polypeptides present in the 1715Δ polypeptides in the heterodimers.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Example 1

A TMMP comprising 1715 (without C-terminal Lys, i.e., 1715Δ having the sequence set forth in SEQ ID NO:58 and the 2380 polypeptide having the sequence set forth in SEQ ID NO:59 (see FIG. 2A-2B) was tested. This TMMP is referred to as the “1715Δ+2380” TMMP. The 1715Δ polypeptide (SEQ ID NO:58) includes: i) a class I HLA-A heavy chain polypeptide of the A02: 01 allele; ii) two copies of a variant IL-2 (H16A; F42A) MOD; and iii) an IgG1 Fc polypeptide comprising L234A and L235A substitutions. The 2380 polypeptide (SEQ ID NO:59) comprises: i) the WT1 peptide WT1 (37-45); and ii) a β2M polypeptide. TMMP 1715Δ+2380 is a double disulfide-linked heterodimer: a) a first disulfide linkage is between: i) the Cys present in the linker between the WT1 peptide and the β2M chain in the 2380 polypeptide; and ii) the Cys introduced by the Y84C substitution in the class I heavy chain present in the 1715Δ polypeptide; and b) a second disulfide linkages is between: i) the Cys introduced by the R12C substitution in the β2M polypeptide present in the 2380 polypeptide; and ii) the Cys introduced by the A236C substitution in the class I heavy chain present in the 1715Δ polypeptide. See FIG. 3A. Further, the 1715Δ-2380 TMMP spontaneously forms a homodimer comprising two copies of the 1715Δ+2380 heterodimer, linked by 2 disulfide bonds between the IgG1 Fc polypeptides present in each of the 1715Δ polypeptides. See FIG. 3B.

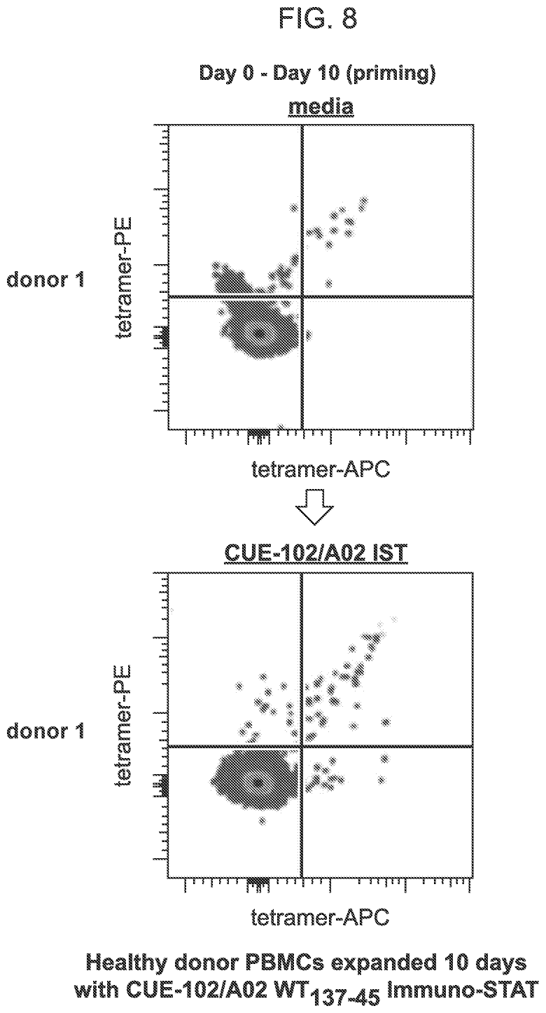

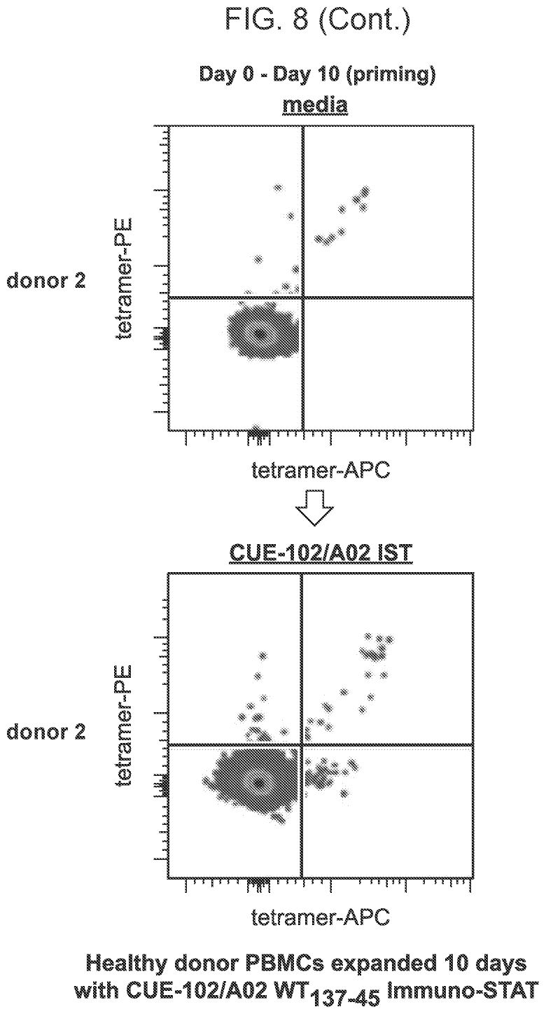

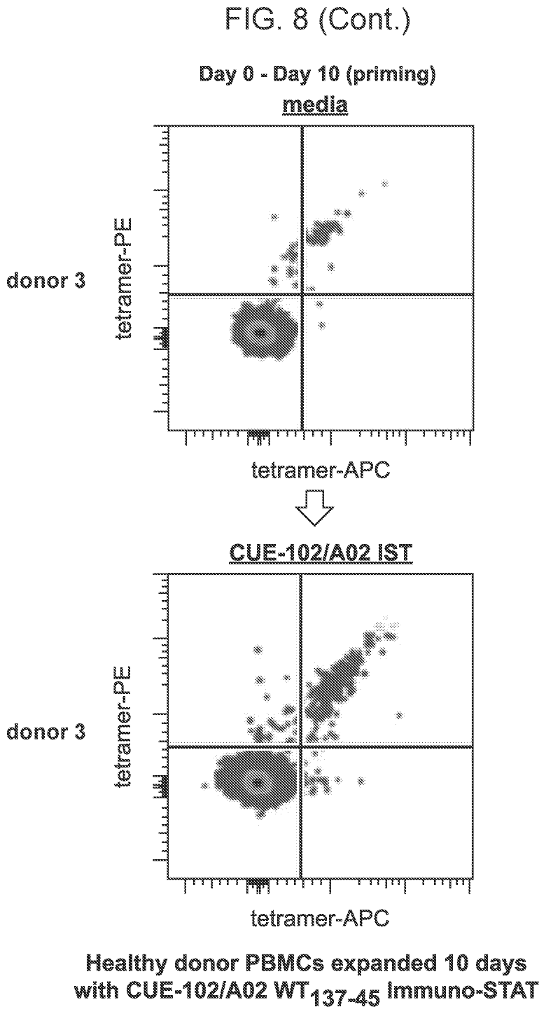

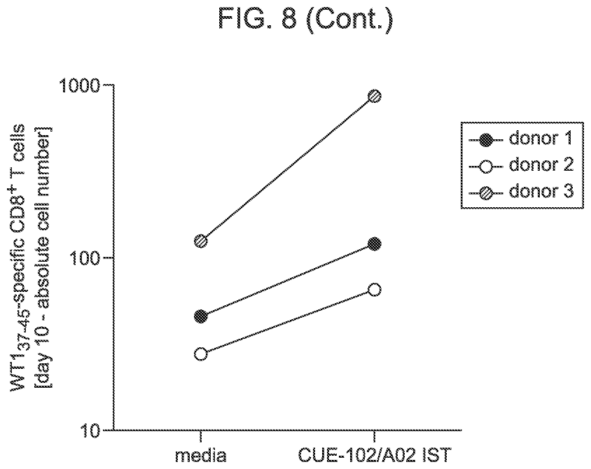

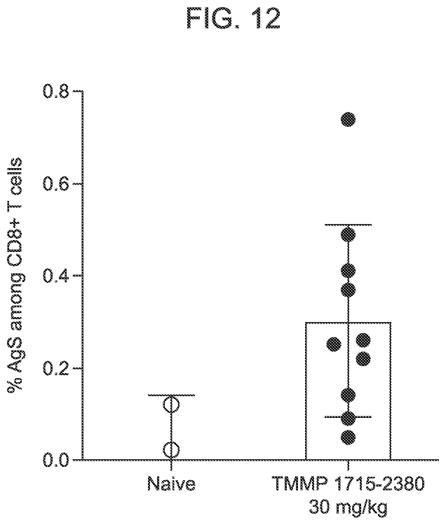

The in vitro effect of a homodimer comprising two TMMPs (1715Δ+2380) on PBMCs is discussed in Example 1 and the data is shown in FIGS. 8-11. The in vivo effect of the same homodimer is discussed in Examples 2-6 below, and the data are shown in FIGS. 12-16. In the Examples and Figures, the homodimer of TMMP 1715Δ+2380 is also referred to below as “CUE-102.” “CUE-102/A02 WT₃₇₋₄₅ Immuno-STAT.” “CUE-102/A02 WT₃₇₋₄₅ IST.” “CUE-102/A02 IST or “CUE/102/A02.”

As shown in FIG. 8, CUE-102/A02 WT1₃₇₋₄₅-IST induces expansion of WT1₃₇₋₄₅-specific CD8⁺ T cells from unprimed PBMCs. Healthy donor PBMCs were stimulated for 10 days with the CUE-102/A02 WT1₃₇₋₄₅ Immuno-STAT (IST) in Immunocult™ media. Cells cultured in the absence of CUE-102/A02 were used as a negative control. Peptide-specific CD8⁺ T cells were detected by flow cytometry upon staining with WT1₃₇₋₄₅-specific tetramers.

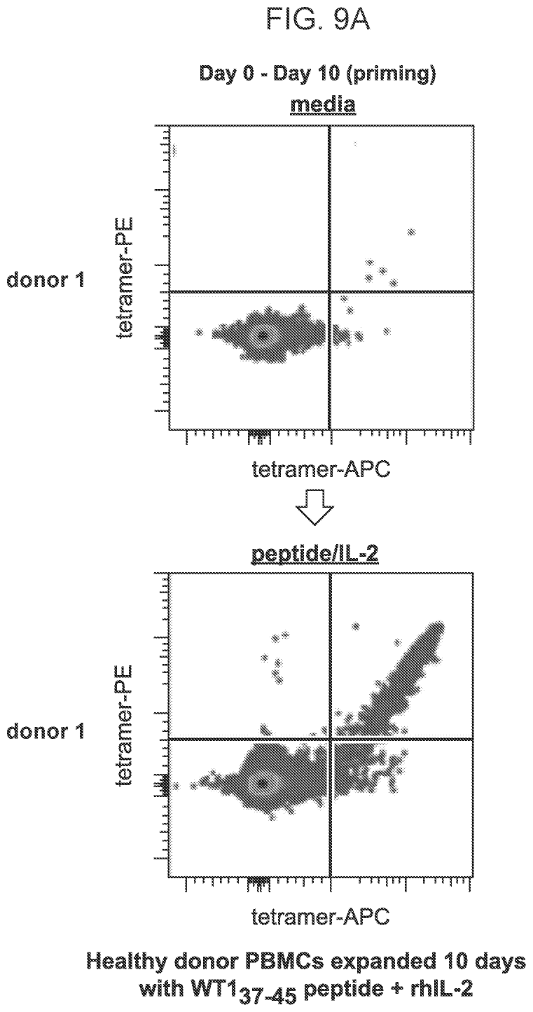

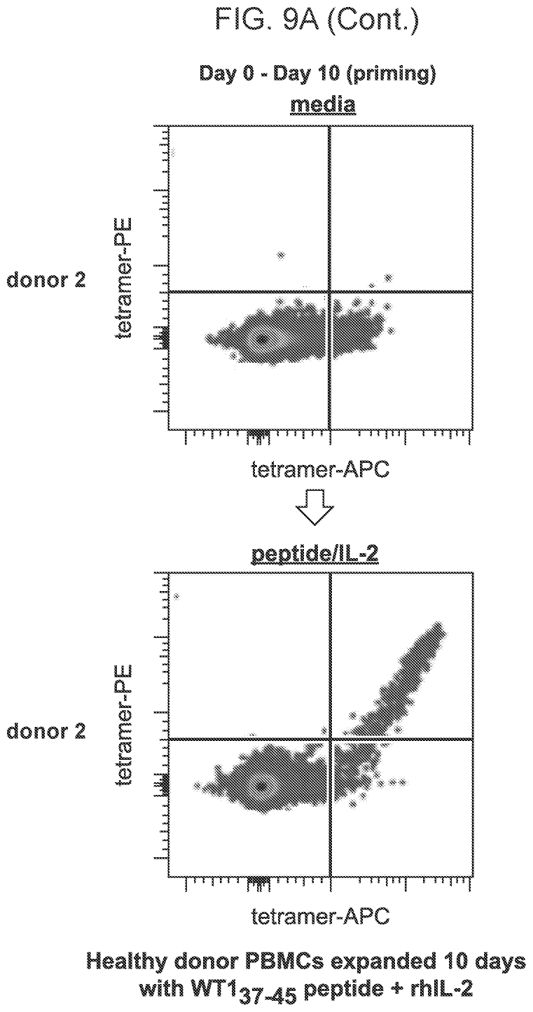

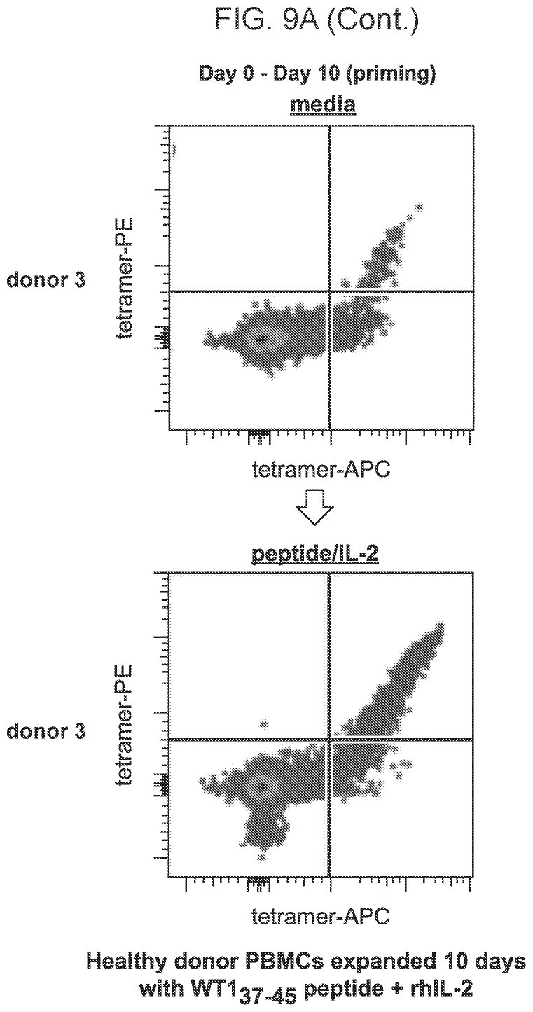

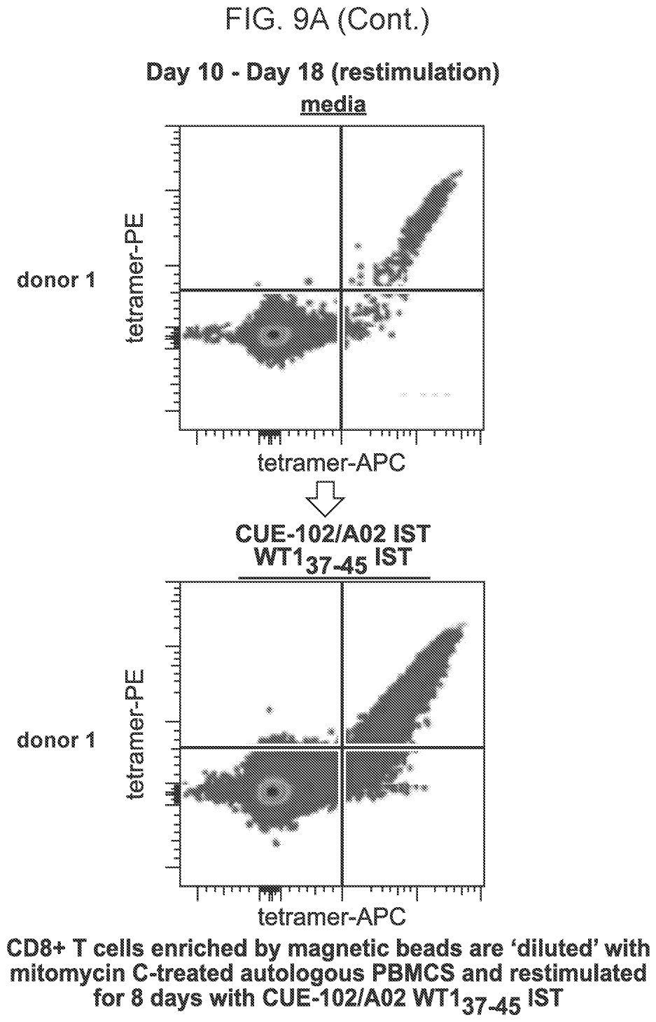

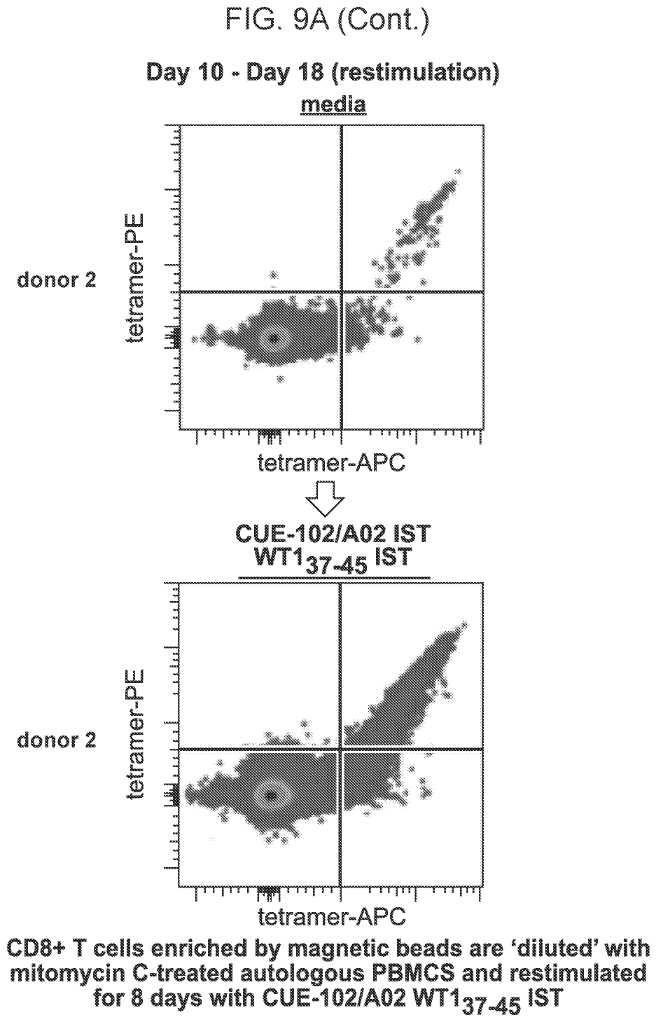

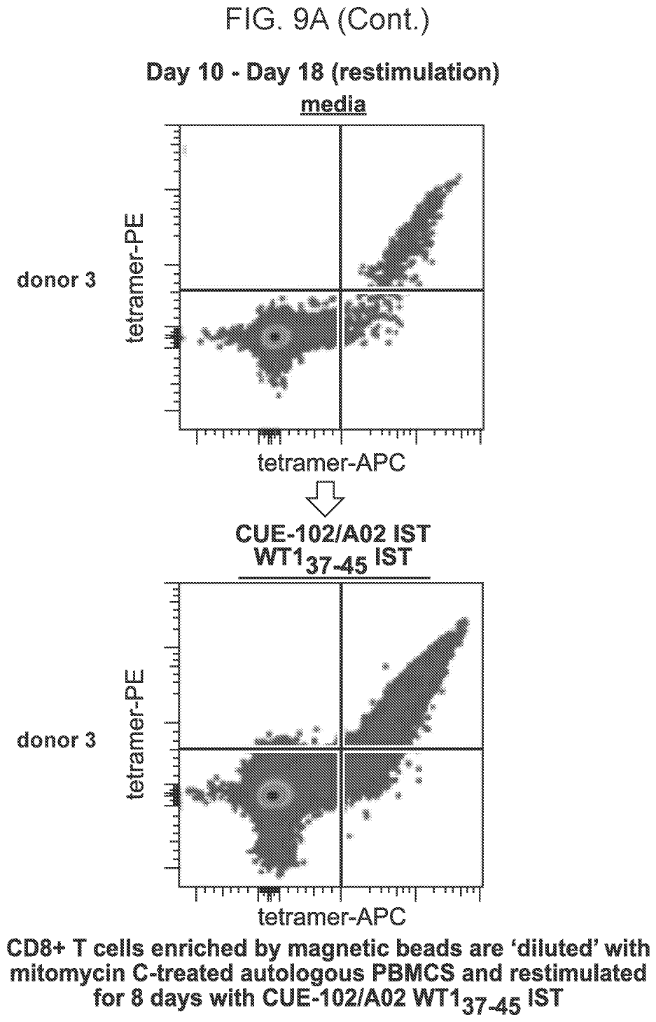

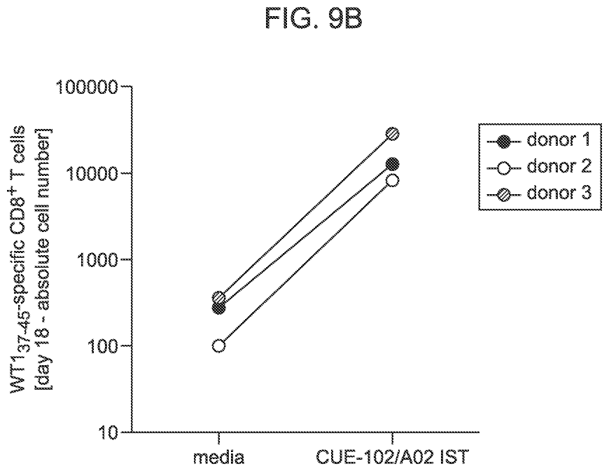

As shown in FIG. 9A-9B, CUE-102/A02 WT1₃₇₋₄₅ IST induces expansion of WT1₃₇₋₄₅-specific CD8⁺ T cells from primed PBMCs. Healthy donor PBMCs were primed for 10 days with WT1₃₇₋₄₅ peptide in the presence of recombinant human IL-2. CD8⁺ T cells were then enriched by magnetic separation and restimulated with the CUE-102/A02 WT1₃₇₋₄₅ IST in Immunocult™ media in the presence of mitomycin C-treated autologous PBMCs for 8 days. Cells restimulated in the absence of CUE-102/A02 were used as a negative control. Peptide-specific CD8⁺ T cells were detected by flow cytometry upon staining with WT1₃₇₋₄₅-specific tetramers.

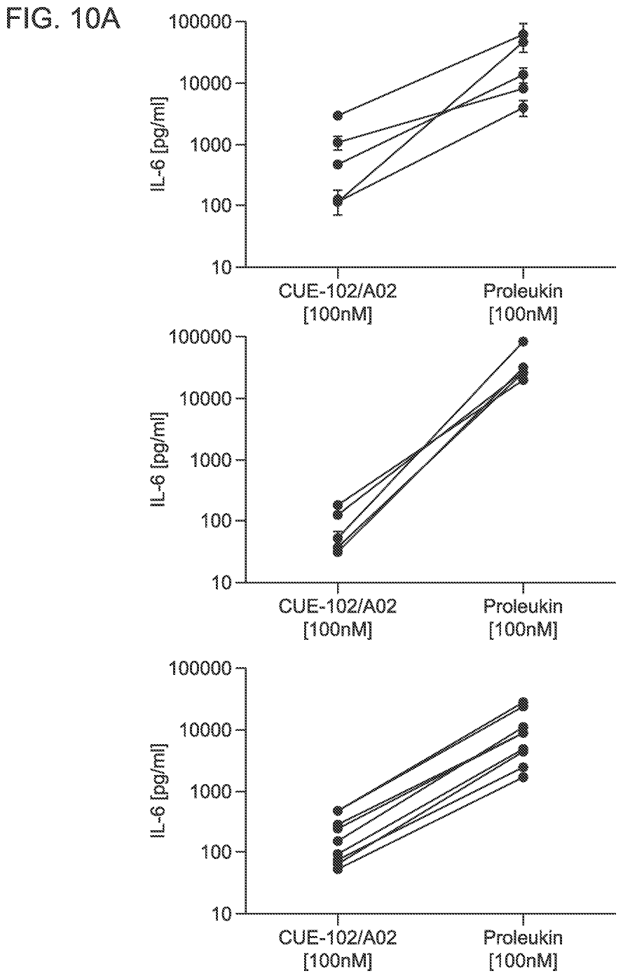

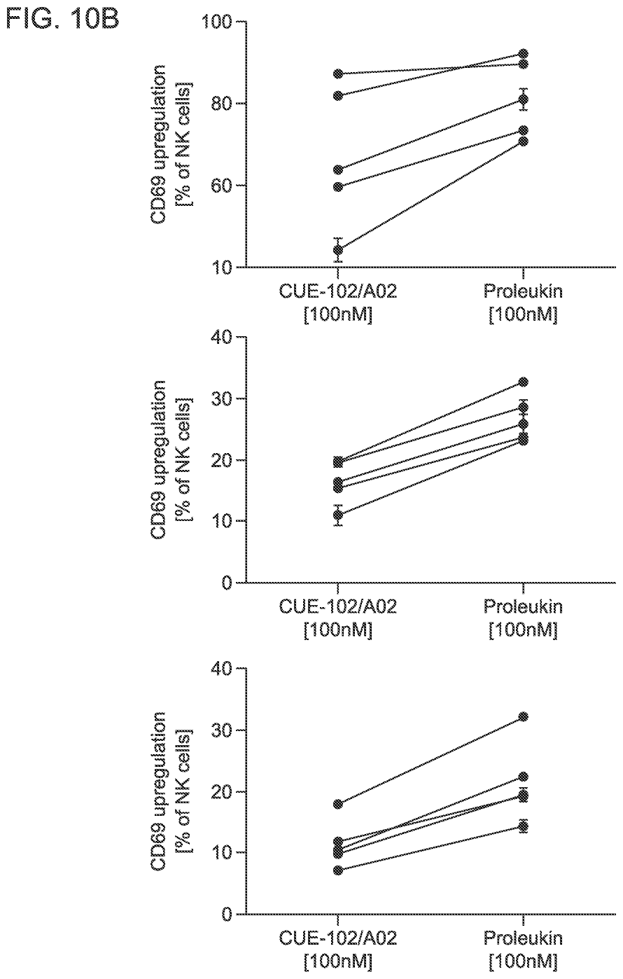

As shown in FIG. 10A-10B, the reduced affinity IL-2-containing CUE-102/A02 WT1₃₇₋₄₅ IST mitigates the risk associated with systemic IL-2 activation, compared to wild-type IL-2. Five healthy donor PBMCs were stimulated with Proleukin® (IL-2), or CUE-102/A02 WT1₃₇₋₄₅IST, in Immunocult™ media for 18 hours. Upon stimulation, supernatants were harvested, and levels of TNF-α, IL-6, and IFN-γ were assessed by immunoassay (FIG. 10A). NK cell, CD4⁺ T cell, and CD8⁺ T cell CD69 upregulation was assessed by flow cytometry on cells from the same culture wells (FIG. 10B). Cells cultured in the absence of Proleukin® (IL-2), or CUE-102/A02 WT1₃₇₋₄₅ IST were used as negative control.

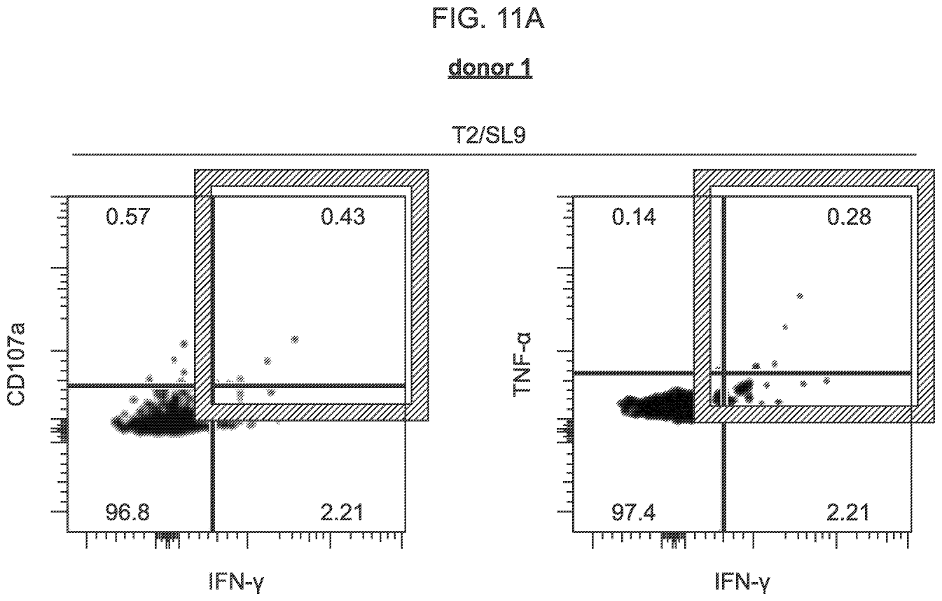

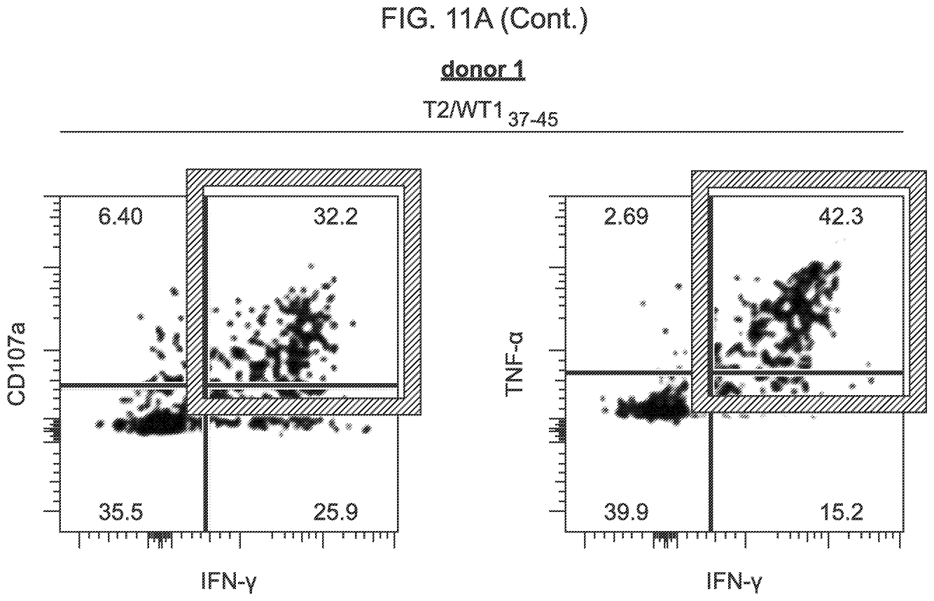

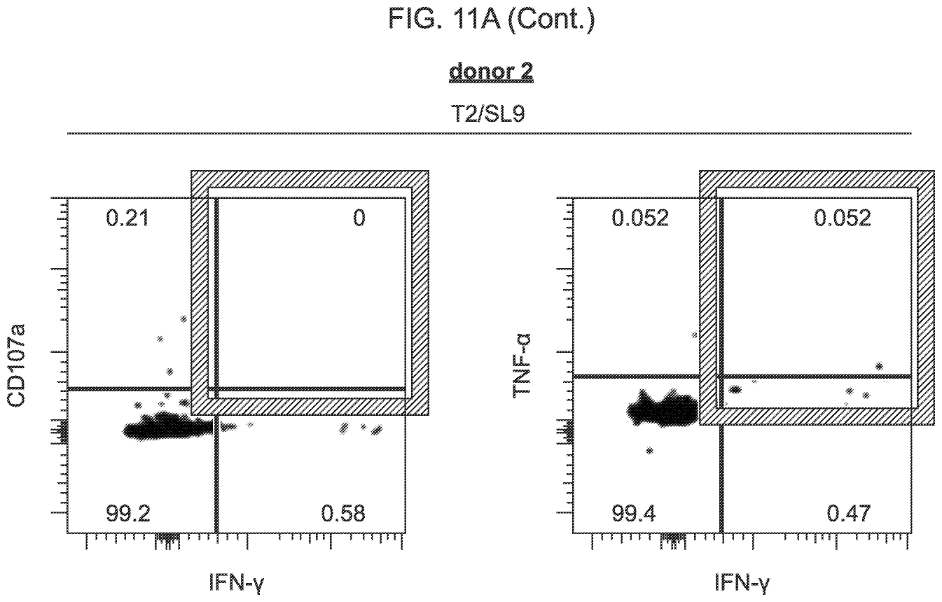

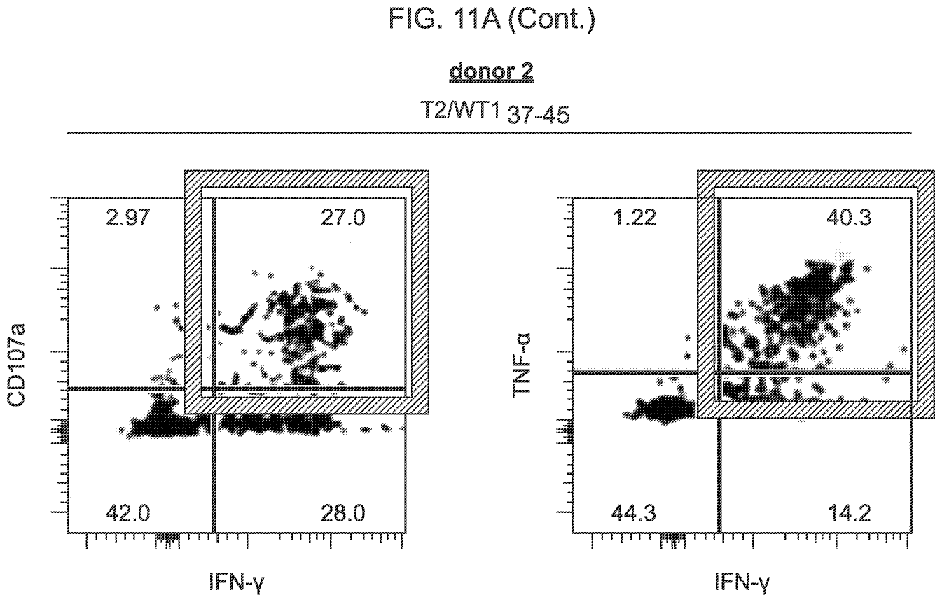

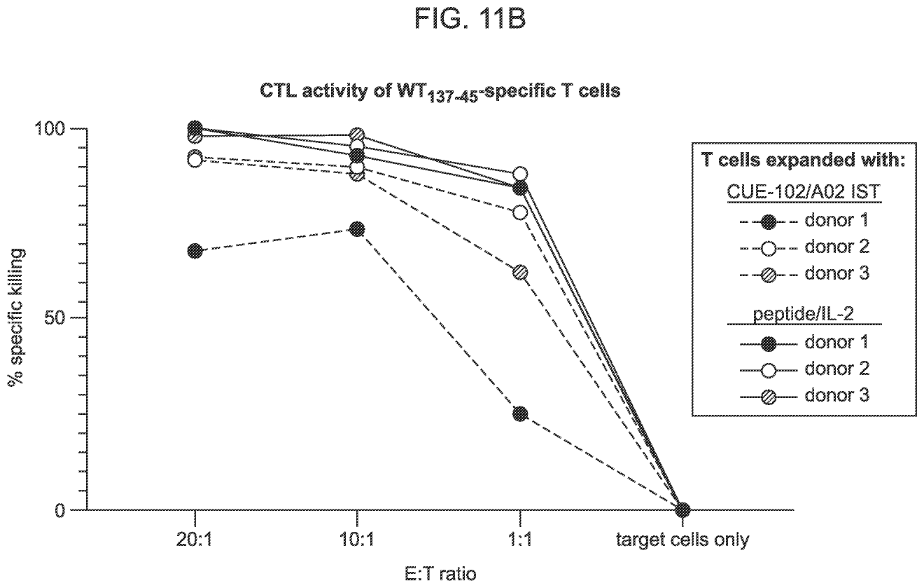

As shown in FIG. 11A-11B, CUE-102/A02 WT1₃₇₋₄₅ IST-expanded T cells are polyfunctional CTLs that recognize and kill WT1₃₇₋₄₅peptide-presenting target cells. Healthy donor PBMCs were primed for 10 days with WT1₃₇₋₄₅peptide in the presence of recombinant human IL-2 and expanded for 8 days with WT1₃₇₋₄₅ peptide or with CUE-102/A02 WT1₃₇₋₄₅ IST in ImmunocultTM media in the presence of mitomycin C-treated autologous PBMCs. WT1₃₇₋₄₅-specific CD8⁺ T cells were enriched by magnetic bead-based separation using WT₁₃₇₋₄₅-specific PE-labeled tetramers. FIG. 11A: CUE-102/A02 WT1₃₇₋₄₅ IST-expanded WT1₃₇₋₄₅-specific T cells expressed effector molecules IFN-γ and TNF-α; and up-regulated the degranulation marker CD107a upon 4 hours of interaction with target T2 cells pulsed with the cognate WT1₃₇₋₄₅peptide, but not with a control, irrelevant peptide (SL9). FIG. 11B: Expanded WT₁₃₇₋₄₅-specific T cell killed cognate WT1₃₇₋₄₅ peptide-pulsed T2 cells, but not control peptide-pulsed T2 cells, in overnight cultures performed at different T cell effector: target cell ratios. Specific killing was assessed by flow cytometry comparing the ratio of viable T2 cell pulsed with cognate peptide vs. control peptide upon overnight culture.

### Example 2

Naïve HLA-A2 (AAD) transgenic mice were dosed intravenously once weekly with 30 mg/kg of CUE-102 (a homodimer comprising two TMMPs 1715Δ+2380 as described above in Example 1) for a total of three doses. The first dose consisted of CUE-102 generated from a transiently transfected cell line, while the subsequent two doses consisted of CUE-102 generated from a stable cell line. The frequency of WT1₃₇₋₄₅-specific CD8+ T cells was then measured in peripheral blood mononuclear cells (PBMCs) 7 days after the last dose. Isolated PBMCs were re-stimulated with WT1₃₇₋₄₅ peptide for 5 hours at 37° C. in the presence of protein transport inhibitors and anti-CD107a antibody to measure degranulation. Cells were then surface stained with WT1₃₇₋₄₅/A02 tetramer, viability dye, and cell surface markers including CD3, CD4, CD8, CD45, CD11b, CD19, and CD44, followed by intracellular staining for IFN-γ. TNF-α, and granzyme B. Antigen-specific cells were detected by analyzing the frequency of tetramer+ cells within the CD8+ T cell population (defined as single, live, CD11b−, CD19−, CD45+, CD3+, CD4−). The frequency of WT1₃₇₋₄₅-specific (“% AgS”) CD8+ T cells in treated mice was found to be greater than that observed in naïve mice that were not treated with CUE-102/A02 WT1₃₇₋₄₅IST (see FIG. 12).

### Example 3

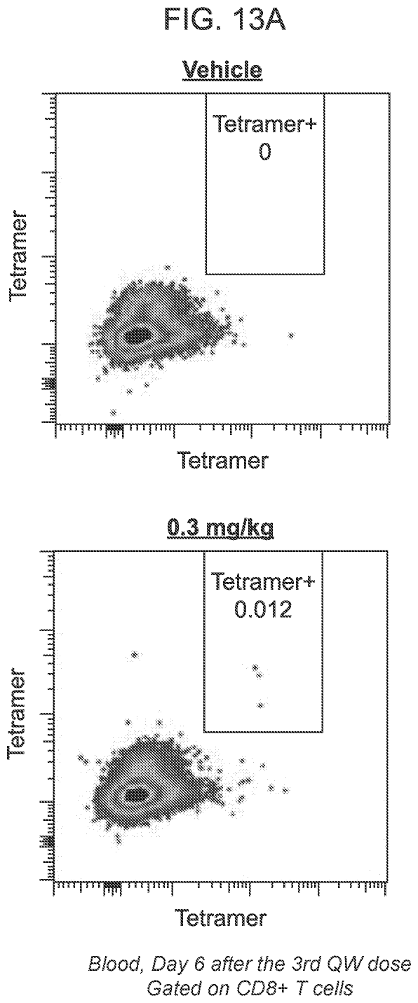

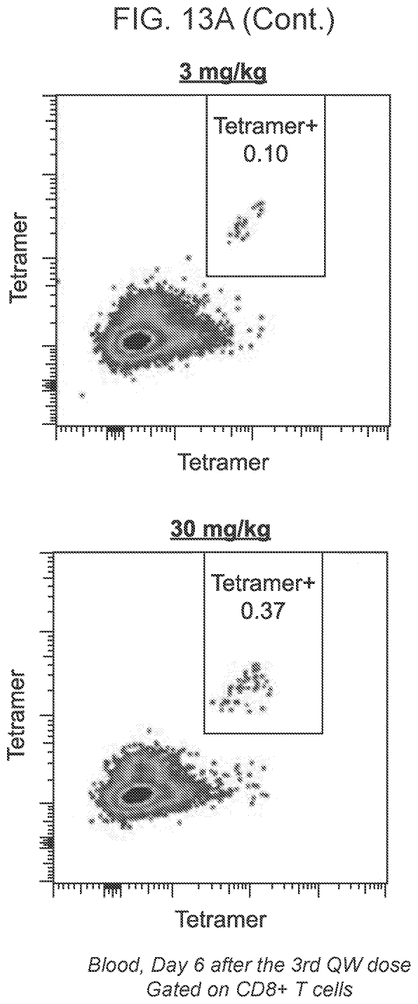

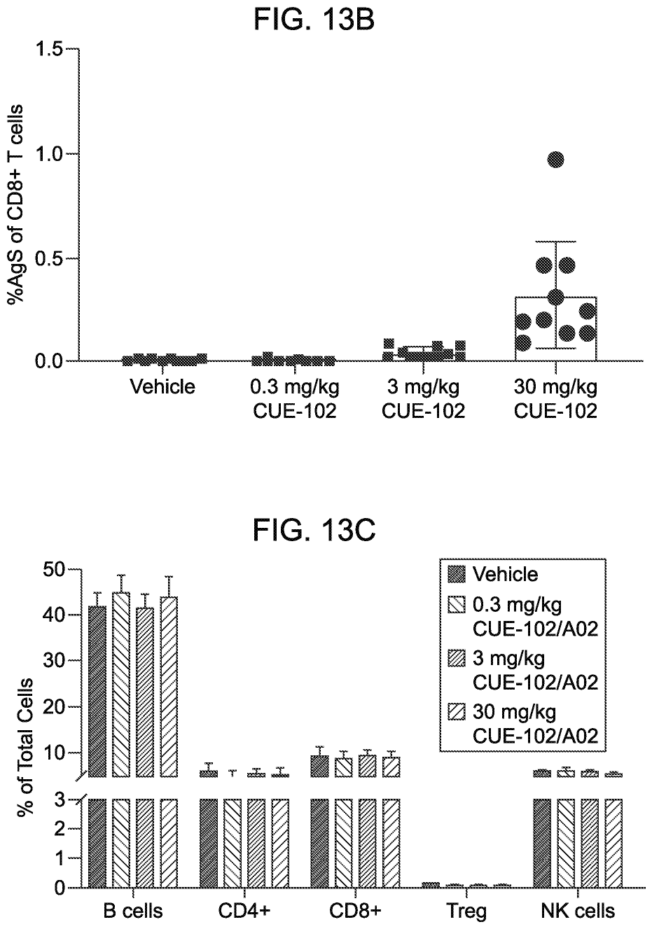

Naïve HLA-A2 (AAD) transgenic mice were dosed intravenously once weekly with 0.3 mg/kg, 3.0 mg/kg, or 30 mg/kg of CUE-102. The frequency of WT1₃₇₋₄₅specific CD8⁺ T cells was then measured in peripheral blood mononuclear cells (PBMCs) 6 days after the third dose. To detect tetramer+ cells and other immune lineages, cells were surface stained with WT1₃₇-45/A02 tetramer, viability dye, and cell surface markers including CD3, CD4, CD8, CD11b, NK1.1, CD19, and CD44, followed by intracellular staining for FoxP3. An increase in the frequency of WT1₃₇₋₄₅tetramer+CD8+ T cells was observed in mice treated with the 30 mg/kg dose of CUE-102 (FIG. 13A and FIG. 13B). Immunophenotyping of major groups of immune cells revealed that CUE-102 treatment did not lead to broad expansion of other immune cells, indicating that CUE-102 selectively expands WT1₃₇₋₄₅-specific CD8+ T cells (FIG. 13C).

### Example 4

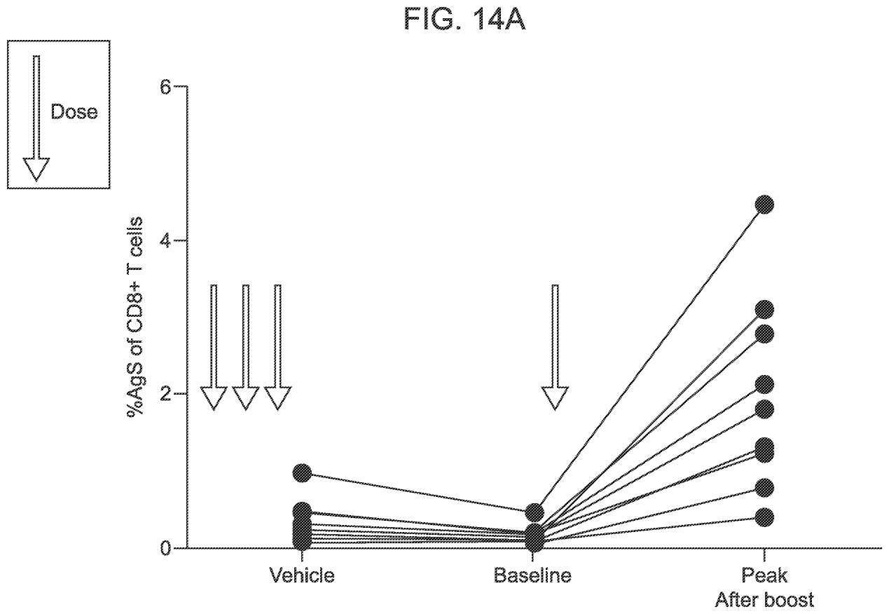

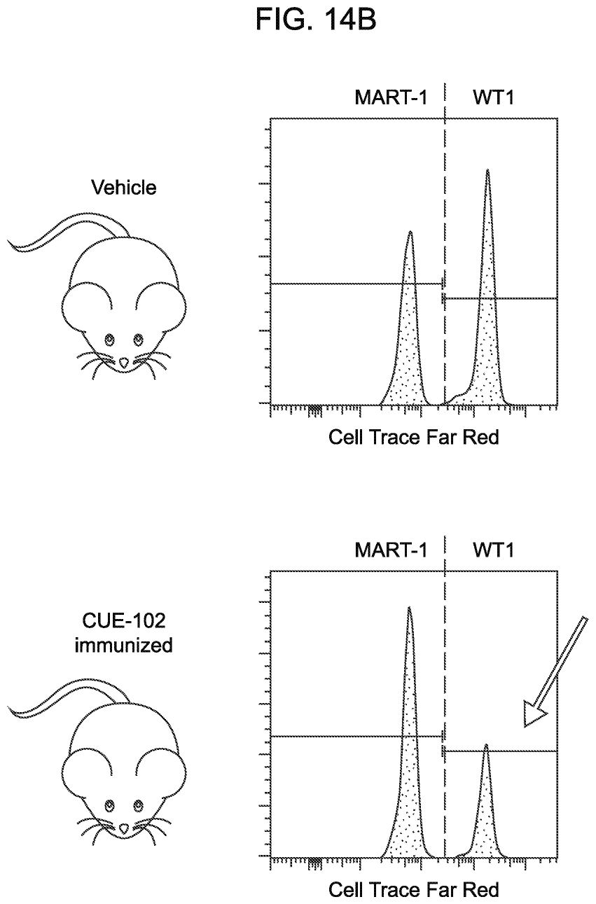

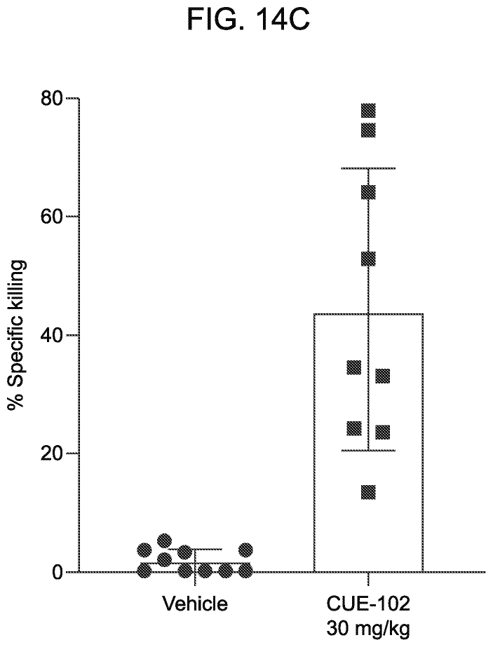

Naive HLA-A2 transgenic mice were immunized with CUE-102 to elicit a population of WT1₃₇₋₄₅-specific CD8+ T cells. Mice were dosed intravenously with 30 mg/kg of CUE-102 given once weekly and expansion of WT1₃₇₋₄₅specific cells was measured in PBMCs 6 days after mice received the 3rd dose of CUE-102. Mice were then rested for an additional three weeks to allow the T cell response to contract, and a baseline measurement was taken to measure frequencies of WT1₃₇₋₄₅-specific CD8+ T cells in the blood, prior to administration of a 4^{th} booster dose. Administration of a 4^{th} dose of CUE-102 further increased the frequency of WT1₃₇₋₄₅specific CD8+ T cells measured in the blood on Day 6 after the 4^{th} dose. (See FIG. 14A.)

The cytotoxic function of WT1-specific cells elicited by immunization with CUE-102 was tested using an assay developed to measure cytotoxic function in vivo. Briefly, syngeneic splenocytes from naïve mice were differentially labeled with Cell Trace Far Red (CTFR) and then pulsed with either specific (e.g., WT1₃₇₋₄₅) peptide (CTFR^{high}) or an irrelevant (eg. MART1) peptide (CTFRlow). CTFRhigh and CTFRlow cells were mixed 1:1 and transferred into vehicle or CUE-102 treated mice. Twenty hours later, the percent specific lysis of the transferred cells was measured in splenocytes from recipient mice and calculated as (1−(average Vehicle Ratio/CUE-102 Ratio))*100 where the ratio is the percent of CTFR^{low}/CTFR^{high} cells. (See FIG. 14B-FIG. 14C). The diminishing epitope-specific peak in CUE-102 treated mice (indicated by the arrow in FIG. 14B) is an indicator of WT1₃₇₋₄₅-specific killing.

### Example 5

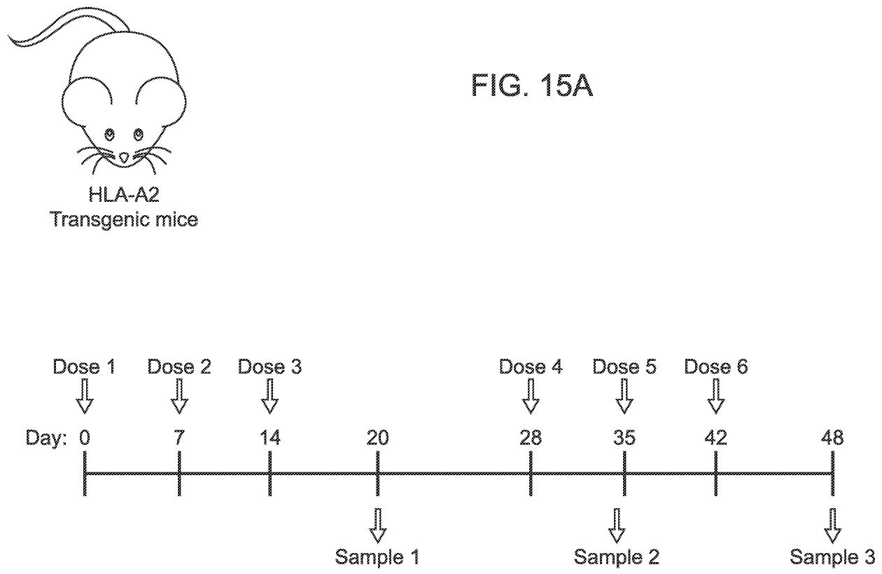

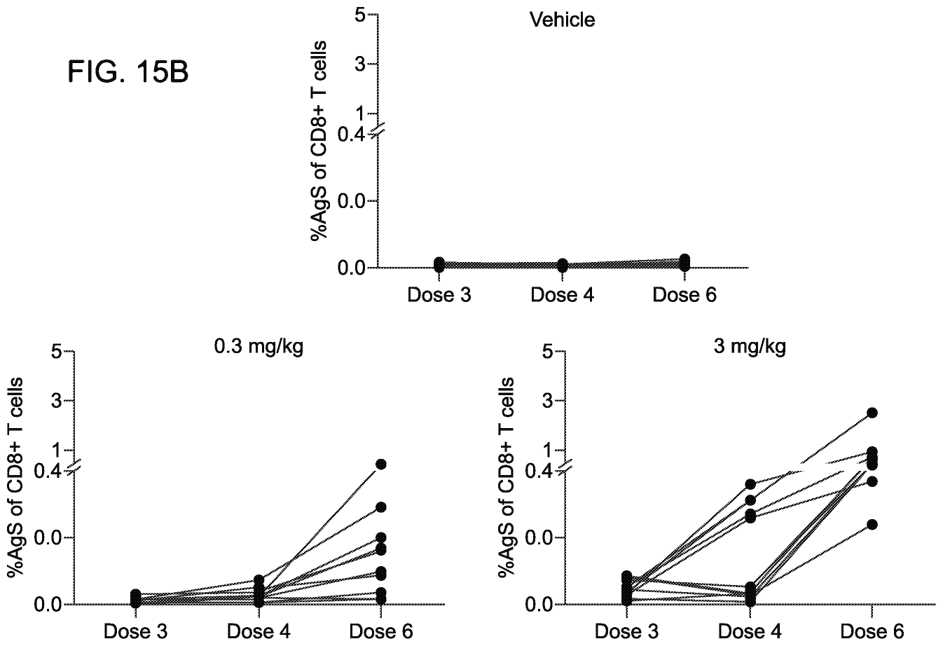

Naive HLA-A2 transgenic mice were dosed intravenously with Vehicle or with CUE-102 at dose levels of 0.3 mg/kg or 3 mg/kg. Mice were given a total of 6 doses of CUE-102. The first 3 doses were administered on a once weekly scheduled, followed by a one-week rest prior to beginning an additional round of 3 once weekly doses. (See FIG. 15A.) The frequency of WT1₃₇₋₄₅-specific CD8+ T cells was then measured in blood 6 days following the 3^{rd}, 4^{th}, and 6^{th} doses of CUE-102. (See FIG. 15B.) Activity of CUE-102 measured by an increase in frequency of WT1₃₇₋₄₅-specific CD8+ T cells was observed following the 4^{th} dose of CUE-102 at the 3 mg/kg but not the 0.3 mg/kg dose level. Both the 0.3 mg/kg and 3 mg/kg dose levels showed activity following the 6^{th} dose of CUE-102, with increased frequencies of WT1₃₇₋₄₅-specific CD8+ T cells observed in some of the animals receiving 0.3 mg/kg CUE-102 and all of the animals receiving 3 mg/kg CUE-102.

### Example 6

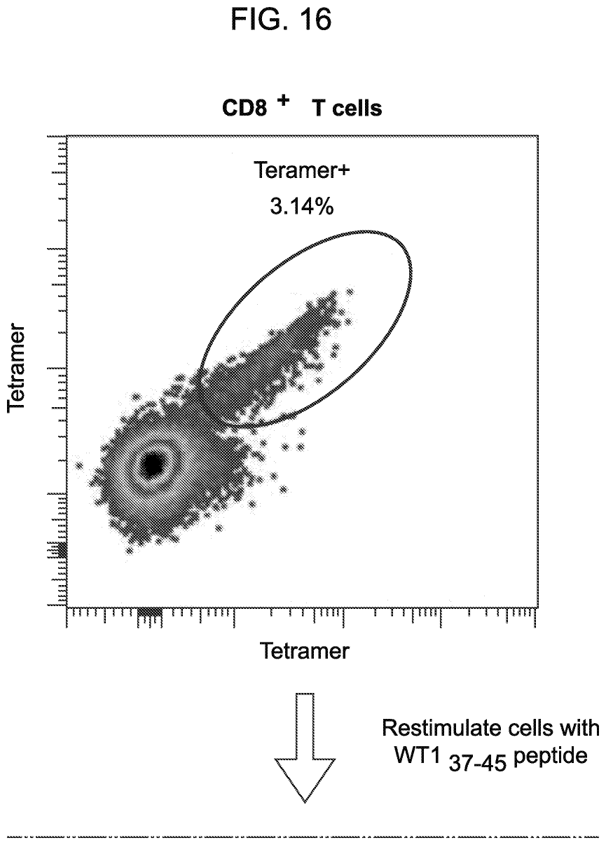

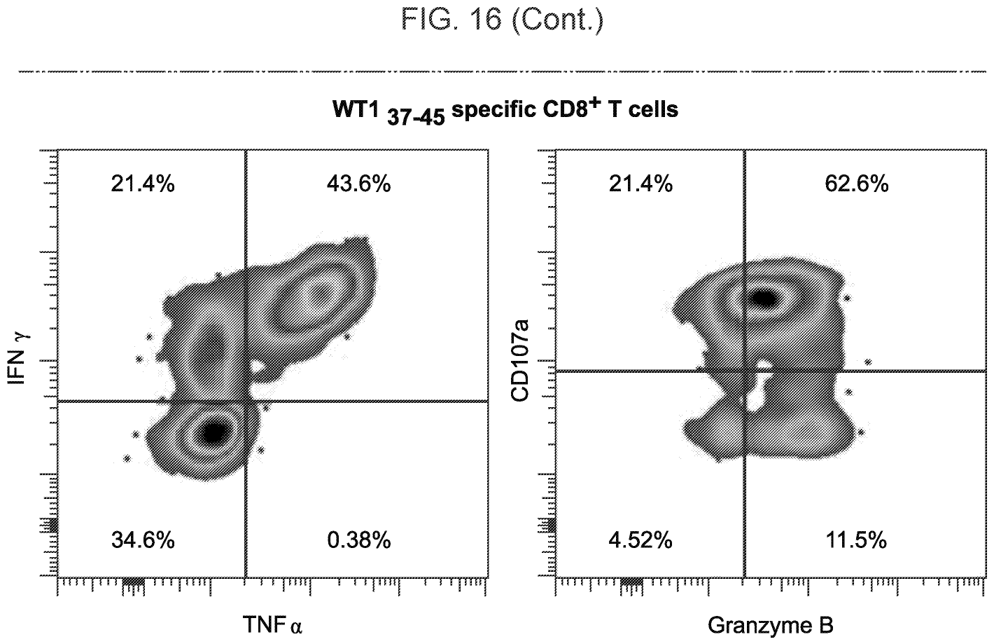

Cells isolated from the spleens of HLA-A2 transgenic mice immunized with a total of 4 doses of CUE-102 were re-stimulated ex vivo with WT1₃₇₋₄₅ peptide for 5 hours at 37° C. in the presence of protein transport inhibitors and anti-CD107a antibody to measure degranulation. Cells were then surface stained with WT1₃₇₋₄₅/A02 tetramer, viability dye, and cell surface markers including CD3, CD4, CD8, and CD44, followed by intracellular staining for IFN-γ, TNF-α, and granzyme B. The majority of tetramer+cells produce multiple cytokines in response to restimulation with WT1₃₇₋₄₅ peptide. (See FIG. 16.)

While the present disclosure has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating a patient having colorectal cancer, gastric cancer, pancreatic cancer, glioblastoma, acute myeloid leukemia (AML), or triple-negative breast cancer that has progressed following treatment with a prior therapy, wherein the method comprises administering to the patient an effective amount of a pharmaceutical composition comprising a TMMP, wherein the TMMP comprises a homodimer comprising two heterodimeric TMMPs, each TMMP comprising: a) a first polypeptide comprising: i) a WT-1 peptide epitope:
and ii) a beta-2 microglobulin (β2M): b) a second polypeptide comprising i) a class I MHC heavy chain polypeptide: ii) at least one variant IL-2 MOD, and iii) an immunoglobulin (Ig) Fc polypeptide.

2. A method according to claim 1, wherein the patient has colorectal cancer that has progressed following treatment with one or more, or all of the following: a fluoropyrimidine, oxaliplatin, irinotecan, bevacizumab.

3. A method according to claim 1, wherein the patient has colorectal cancer associated with a KRAS mutation, and the cancer has progressed following treatment with one or both of cetuximab and panitumumab.

4. A method according to claim 1, wherein the patient has colorectal cancer that has progressed following treatment with regorafenib and/or trifluridine/tipiracil.

5. A method according to claim 1, wherein the patient has colorectal cancer that has progressed following treatment with one, more than one, or all of the following: a fluoropyrimidine, oxaliplatin, irinotecan, bevacizumab, and has progressed following treatment with one or both of cetuximab and panitumumab, and has progressed following treatment with regorafenib and/or trifluridine/tipiracil.

6. A method according to claim 1, wherein the patient has gastric cancer and has MSI-H or high tumor mutational burden, and wherein the cancer has progressed following treatment with one or more immune checkpoint inhibitors.

7. A method according to claim 1, wherein the patient has gastric cancer that is HER2+ and the cancer has progressed in the relapse setting after treatment with a HER2-directed antibody and topoisomerase inhibitor conjugated.

8. A method according to claim 1, wherein the patient has gastric cancer that harbors a NTRK gene fusion and the cancer has progressed following treatment with an Entrectinib class TKI.

9. A method according to claim 1, wherein the patient has gastric cancer that has progressed following treatment with each of an immune checkpoint inhibitor, a HER2-directed antibody and topoisomerase inhibitor conjugated, and an Entrectinib class TKI.

10. A method according to claim 1, wherein the patient has pancreatic cancer, and wherein the cancer has progressed following prior treatment selected from a fluoropyrimidine-based regime, a gemcitabine-based regimen, or both, in either the adjuvant or relapsed setting.

11. A method according to claim 1, wherein the patient has glioblastoma that has progressed following treatment with one, two, or all of the following: surgery, radiation, and temozolomide.

12. A method according to claim 1, wherein the patient has ovarian cancer that has progressed following treatment with one, two, or all of the following: surgery, antibody therapy, carboplatin, and paclitaxel.

13. A method according to claim 1, wherein the patient has AML that has progressed following treatment with one or more, or all of the following: (i) a combination of cytarabine and an anthracycline drug: (ii) a FLT3 inhibitor; (iii) an IDH inhibitor; (iv) gemtuzumab ozogamicin: (v) a BCL-2 inhibitor; and (v) a hedgehog pathway inhibitor.

14. A method according to claim 1, wherein the patient has triple-negative breast cancer that has progressed following treatment with one or more, or all of the following: (i) surgery: (ii) olaparib: (iii) capecitabine (Xeloda); and (iv) pembrolizumab.

15. A method of treating a patient having a cancer according to claim 1, wherein the amount of TMMP homodimer administered is selected from 0.5 to 1 mg/kg body weight, 1 mg/kg body weight to 5 mg/kg, and from 5 mg/kg body weight to 10 mg/kg body weight.

16. A method of treating a patient having a cancer according to claim 1, wherein the amount of TMMP homodimer administered is 1 mg/kg body weight.

17. A method of treating a patient having a cancer according to claim 1, wherein the amount of TMMP homodimer administered is 2 mg/kg body weight.

18. A method of treating a patient having a cancer according to claim 1, wherein the amount of TMMP homodimer administered is 3 mg/kg body weight.

19. A method of treating a patient having a cancer according to claim 1, wherein the amount of TMMP homodimer administered is 4 mg/kg bodyweight.

20. A method of treating a patient having a cancer according to claim 1, wherein the amount of TMMP homodimer administered is 5 mg/kg body weight.

21-43. (canceled)
